(19) United States
(12) Patent Application Publication
(10) Pub. No.: US 2025/0349392 A1
(43) Pub. Date: Nov. 13, 2025

(54) Enhanced Iron-Based Oligomerization of Ethylene Using Machine Learning-Based K-Value Prediction

(71) Applicant: Chevron Phillips Chemical Company LP, The Woodlands, TX (US)

(72) Inventors: Michael S. Webster-Gardiner, Humble, TX (US); Daniel H. Ess, Provo, UT (US); Steven M. Bischof, Spring, TX (US); Brooke L. Small, Kingwood, TX (US); Julie A. Leseberg, Kingwood, TX (US)

(21) Appl. No.: 19/200,394

(22) Filed: May 6, 2025

Related U.S. Application Data
(60) Provisional application No. 63/643,596, filed on May 7, 2024.
(60) Provisional application No. 63/643,606, filed on May 7, 2024.
(60) Provisional application No. 63/643,618, filed on May 7, 2024.

(51) Int. Cl. G16C 20/10; C07C 2/34; G06N 20/20; G16C 20/70

(52) U.S. Cl. CPC G16C 20/10; C07C 2/34; G06N 20/20; G16C 20/70; C07C 2531/28

(57) ABSTRACT

A machine learning model predicts a K value for a new iron ethylene oligomerization catalyst structure, where the α value has not yet been experimentally determined.

### CROSS-REFERENCE TO RELATED APPLICATIONS

This application is a non-provisional patent application claiming the benefit of, and priority to, U.S. Provisional Patent Application No. 63/643,606, filed May 7, 2024, U.S. Provisional Patent Application No. 63/643,596, filed May 7, 2024, U.S. Provisional Patent Application No. 63/643,618, filed May 7, 2024, each of which is incorporated by reference herein in its entirety.

### TECHNICAL FIELD

The present disclosure relates to iron-based catalysts for the oligomerization of ethylene, and more particularly, to using machine learning to identify new catalyst structures through prediction of catalyst K values.

### BACKGROUND

The development of improved catalysts can be a daunting task and often requires laborious trial and error synthetic work. For example, the development of new homogeneous Fe catalysts for ethylene oligomerization to produce α-olefins includes the slow synthetic development of new ligand species. Another major impediment in the development of new homogeneous Fe catalysts for ethylene oligomerization is the prediction of propagation versus termination rates that control the α-olefin distribution. Because the transition states for propagation versus termination are generally separated by a difference of less than one kcal/mol in energy, this selectivity may not be accurately predicted by standard computational methods. New methods are needed for predicting these parameters which are useful in systems with such small transition state energies.

### SUMMARY

This disclosure provides for new methods, systems, devices, and computer readable media for identifying iron-based catalysts for the oligomerization of ethylene using machine learning-based K value prediction.

In aspects, a method can include: inputting a set of reaction conditions and a new iron ethylene oligomerization catalyst structure including a ligand to a random forest machine learning regressor model, wherein the random forest machine learning regressor model is trained on a data set including multi-dimensional features for tested iron ethylene oligomerization catalyst structures, wherein the multi-dimensional features include experimental K values, physical features, molecular features, and connective steric factors for each of the tested iron ethylene oligomerization catalyst structures; predicting, by the random forest machine learning regressor model, a predicted K value for the new iron ethylene oligomerization catalyst structure for the set of reaction conditions; and after predicting, experimentally determining an experimental K value for the new iron ethylene oligomerization catalyst structure under the set of reaction conditions.

In aspects, a system can include: a device including memory coupled to at least one processor, the memory having instructions that cause the at least one processor to: input a set of reaction conditions and a new iron ethylene oligomerization catalyst structure including a ligand to a random forest machine learning regressor model, wherein the random forest machine learning regressor model is trained on a data set including multi-dimensional features for tested iron ethylene oligomerization catalyst structures, wherein the multi-dimensional features include experimental K values, physical features, molecular features, and connective steric factors for each of the tested iron ethylene oligomerization catalyst structures; run the random forest machine learning regressor model to predict a predicted K value for the new iron ethylene oligomerization catalyst structure under the set of reaction conditions, wherein the predicted K value is obtained before an experimental K value is obtained for the new iron ethylene oligomerization catalyst structure.

In aspects, a computer-readable medium having instructions stored thereon, that when executed by at least one processor causes the at least one processor to perform operations including: input a set of reaction conditions and a new iron ethylene oligomerization catalyst structure comprising a ligand to a random forest machine learning regressor model, wherein the random forest machine learning regressor model is trained on a data set comprising multi-dimensional features for tested iron ethylene oligomerization catalyst structures, wherein the multi-dimensional features comprise experimental K values, physical features, molecular features, and connective steric factors for each of the tested iron ethylene oligomerization catalyst structures; run the random forest machine learning regressor model to predict a predicted K value for the new iron ethylene oligomerization catalyst structure under the set of reaction conditions, wherein the predicted K value is obtained before an experimental K value is obtained for the new iron ethylene oligomerization catalyst structure.

In an aspect, a method can include: determining a set of multi-dimensional features for first iron ethylene oligomerization catalyst structures; inputting the set of multi-dimensional features to a machine learning model trained to predict

$$K ( \frac{{C}_{12}}{{C}_{10}} )$$

values for second iron ethylene oligomerization catalyst structures based on the multi-dimensional features; predicting, using the machine learning model and based on the set of multi-dimensional features, first

$$K ( \frac{{C}_{12}}{{C}_{10}} )$$

values; identifying, using the machine learning model and based on the set of multi-dimensional features, an ethylene oligomerization catalyst structure as a most influential of the second iron ethylene oligomerization catalyst structures to the first

$$K ( \frac{{C}_{12}}{{C}_{10}} )$$

values; and outputting the first

$$K ( \frac{{C}_{12}}{{C}_{10}} )$$

values and an indication that the ethylene oligomerization catalyst structure is the most influential of the second iron ethylene oligomerization catalyst structures to the first

$$K ( \frac{{C}_{12}}{{C}_{10}} )$$

values.

In another aspect, a device can include: memory coupled to at least one processor, the at least one processor configured to: determine a set of multi-dimensional features for first iron ethylene oligomerization catalyst structures, wherein the set of multi-dimensional features comprises physical features corresponding to reaction conditions, comprises molecular features, and comprises connective steric factors; input the set of multi-dimensional features to a machine learning model trained to predict

$$K ( \frac{{C}_{12}}{{C}_{10}} )$$

values measuring selectivity for propagation versus termination during oligomerization catalysis for second iron ethylene oligomerization catalyst structures based on the multi-dimensional features; predict, using the machine learning model and based on the set of multi-dimensional features, first

$$K ( \frac{{C}_{12}}{{C}_{10}} )$$

values; identify, using the machine learning model and based on the set of multi-dimensional features, an ethylene oligomerization catalyst structure as a most influential of the second iron ethylene oligomerization catalyst structures to the first

$$K ( \frac{{C}_{12}}{{C}_{10}} )$$

values; and output the first

$$K ( \frac{{C}_{12}}{{C}_{10}} )$$

values and an indication that the ethylene oligomerization catalyst structure is the most influential of the second iron ethylene oligomerization catalyst structures to the first

$$K ( \frac{{C}_{12}}{{C}_{10}} )$$

values.

In another aspect, a computer-readable medium can: store instructions for identifying ligands for iron-based oligomerization of ethylene, that when executed by at least one processor cause the at least one processor to perform operations including: determining a set of multi-dimensional features for first iron ethylene oligomerization catalyst structures, wherein the set of multi-dimensional features comprises physical features corresponding to reaction conditions, comprises molecular features, and comprises connective steric factors; inputting the set of multi-dimensional features to a machine learning model trained to predict

$$K ( \frac{{C}_{12}}{{C}_{10}} )$$

values measuring selectivity for propagation versus termination during oligomerization catalysis for second iron ethylene oligomerization catalyst structures based on the multi-dimensional features; predicting, using the machine learning model and based on the set of multi-dimensional features, first

$$K ( \frac{{C}_{12}}{{C}_{10}} )$$

values; identifying, using the machine learning model and based on the set of multi-dimensional features, an ethylene oligomerization catalyst structure as a most influential of the second iron ethylene oligomerization catalyst structures to the first

$$K ( \frac{{C}_{12}}{{C}_{10}} )$$

values; and outputting the first

$$K ( \frac{{C}_{12}}{{C}_{10}} )$$

values and an indication that the ethylene oligomerization catalyst structure is the most influential of the second iron ethylene oligomerization catalyst structures to the first

$$K ( \frac{{C}_{12}}{{C}_{10}} )$$

values.

These and other aspects, embodiments, and improvements are described more fully herein.

### BRIEF DESCRIPTION OF THE DRAWINGS

FIG. 1 is an exemplary process for machine learning used for the prediction of iron-based ethylene oligomerization catalyst K values in accordance with one embodiment.

FIGS. 2A to 2AD illustrate examples of iron-based ethylene oligomerization catalyst structures used in a data set to train and test the machine learning model of FIG. 1.

FIG. 3A illustrates an example complex of the iron-based ethylene oligomerization catalyst of FIG. 2K.

FIG. 3B illustrates example molecules for the C positions of the phenyl ring of the complex of FIG. 3A.

FIG. 3C illustrates an example length of a methylbenzene molecule of the complex of FIG. 3A.

FIG. 3D illustrates an example width of a methylbenzene molecule of the complex of FIG. 3A.

FIG. 3E illustrates an example depth of a methylbenzene molecule of the complex of FIG. 3A.

FIG. 4 illustrates example molecules for generating a characteristic size of substitution groups on polydentate ligand arms of the iron-based ethylene oligomerization catalysts of FIGS. 2A to 2AD.

FIG. 5 is a schematic diagram of machine learning for the process of FIG. 1 in accordance with one embodiment.

FIG. 6 is a flowchart illustrating a process utilizing K value prediction in accordance with one embodiment.

FIG. 7 is a diagram illustrating an example of a computer system that may be used in implementing embodiments of the present disclosure.

FIG. 8 is a flowchart illustrating a process for predicting K values for new catalyst structures for ethylene oligomerization in accordance with one embodiment.

### DETAILED DESCRIPTION

“K value” refers to a dimensionless number that indicates a distribution of α-olefins produced by a catalyst under a combination of reaction conditions for the catalyzed oligomerization of ethylene. The α value can be expressed as (moles Cₙ₊₂/moles Cₙ) which is a measure of the selectivity for propagation versus termination during oligomerization of ethylene. Examples of K values disclosed herein include

$$K ( \frac{{C}_{12}}{{C}_{10}} )$$

values and

$$K ( \frac{{C}_{14}}{{C}_{12}} )$$

values.

“New iron ethylene oligomerization catalyst structure” and its variants such as “new catalyst” and “new catalyst structure” refer to a catalyst structure for which a K value has not been experimentally determined before inputting the catalyst structure into the machine learning model that predicts a K value for the structure.

“Tested iron ethylene oligomerization catalyst structure” refers to a catalyst structure for which at least one K value associated with a set of reaction conditions has previously been experimentally determined and characterizes the catalyst structures used to train the machine learning model that predicts a K value for another, new, structure or a K value for the same catalyst structure under another set of reaction conditions that have not been experimentally tested for the catalyst structure.

K values for iron ethylene oligomerization catalyst structures are usually determined experimentally. Thus, if a new catalyst structure, a new ligand for a catalyst structure, or new substitutions of groups on a ligand are to be developed, the new structure must be synthesized and the α value experimentally determined. Because a myriad of new catalyst structures are possible, experimentally determining K values for them all is constrained by time, resources, and the lack of predictability of whether a particular synthesis would even lead to an effective catalyst. The machine learning model disclosed herein predicts a K value for a new iron ethylene oligomerization catalyst structure, where the K value has not yet been experimentally determined. Procedures for catalyst development in the field are significantly affected since the predicted K value can be used to identify a potentially effective new catalyst structure without requiring physical synthesis and testing of the new catalyst structure to determine the K value. Testing of the new catalyst structure for an experimental K value after obtaining the predicted K value significantly changes the experimental testing to a validation, to validate the machine learning model's K value, instead of being a trial and error endeavor to find an unknown K value that may or may not be suitable for ethylene oligomerization. By predicting K values as disclosed herein, the endeavor of iron ethylene oligomerization catalyst development can be flipped on its head, where K values are predicted before experimentation, and then, after a predicted K value indicates that a catalyst structure may be effective for ethylene oligomerization, the K value of the catalyst structure is experimentally obtained to determine how the catalyst structure could be used for ethylene oligomerization.

Linear α-olefins (i.e., 1-alkenes), specifically C₄ to C₁₈, are important chemical precursors used in the production of several relevant commodities such as polyethylene, plasticizers, lubricants, surfactants, and other materials. Fe-based catalysts are highly desirable due to the abundant, low-cost, and non-toxic nature of iron. Iron oligomerization catalysts engender high reactivity and enable significant diversity of ligand architectures that can be used to control reaction selectivity. A major impediment in the design of novel Fe-based ethylene oligomerization catalysts is the prediction of the α-olefin selectivity distribution.

The distribution of α-olefins produced is typically described as the K value (expressed as (moles Cₙ₊₂/moles Cₙ)) which is a measure of the selectivity for propagation versus termination during oligomerization. This value, which is mathematically described as a constant, often shows small amounts of drift over the total product range and is therefore often reported as a ratio of C₁₂/C₁₀ or C₁₄/C₁₂. Propagation-termination selectivity is controlled by the energy difference between transition states for Fe-alkyl ethylene insertion for propagation and termination by β-hydrogen transfer. Based on experimentally reported K values and statistical rate theory, the energy difference between these transition states is often less than 1 kcal/mol. Thus, predicting the K values for ethylene oligomerization is outside the reach of density functional theory (DFT) and generally outside the reach of CCSD(T) (coupled cluster singles and doubles) and DLPNO-CCSD(T) (domain-based local pair natural orbital) that can be applied to moderate to large size catalysts.

In one or more embodiments, a machine learning (ML)-based model built using experimental data and molecular structure features can provide the necessary sub-kcal/mol accuracy to enable the prediction of K values. For example, the machine learning model may be used to target new ligand structures to generate desired K values, such as, by changing the phosphine andaryl imine substitution, control of the K value can be tuned between 0.57 and 0.72. In addition to the model being based on experiments rather than DFT computed data, this type of approach has the advantage of no significant computational cost to predict the K values of new possible ligands. The accuracy of the enhanced K value prediction herein also is improved with respect to DFT, CCSD(T), and DLPNO-CCSD(T) techniques (e.g., to a sub-kcal/mol accuracy for the K values).

In one or more embodiments, the ML can allow for determining K value energetics (e.g., propagation and termination rates) with sub-kcal accuracy (e.g., an improved K value accuracy) without the ongoing use of energy and time intensive existing DFT techniques.

In one or more embodiments, the predicted K values herein can be interpolative rather than generative based on experimental K values. The machine learning model can be built using selectivity values and molecular descriptors (e.g., features) that do not rely on information generated from quantum-chemical calculations, such as atomic charges or vibrational frequencies. Physical features such as reaction temperature and reagent loading are considered in the model.

In one or more embodiments, the experimental K values can include an experimental K(C₁₂/C₁₀) value data set using 116 unique polydentate (mostly tridentate) Fe catalysts. For example, a set of example tridentate Fe catalysts bearing various ligand backbones featuring a diverse set of substituents on the ligand arms near the Fe center can be used. This dataset includes N, O, S, and P direct coordination with the Fe metal center, and pyridine-bisimine, α-diimine, phenanthroline, iminopyridine, and other derivative ligands.

In one or more embodiments, the 116 catalysts all can have an associated K value, and some may have multiple K values corresponding to different respective reaction conditions (e.g., catalyst loading (including co-catalyst loading), cocatalyst identity, ethylene pressure, time, and reaction temperature). The data set can encompass a total of 257 K values for these 116 different catalysts. A few values were reported as K(C₁₄/C₁₂) and converted to K(C₁₂/C₁₀) values through the linear scaling using Equation (1):

$$K ( \frac{{C}_{12}}{{C}_{10}} ) = K ( \frac{{C}_{14}}{{C}_{12}} ) \times 0.953 . \qquad ( 1 )$$

The scaling according to Equation (1) is justified based on experimental K values for different carbon fractions (e.g., C₄-C₂₀) measured using a Fe pendant donor diimine (Fe(PDD)) catalyst. Although this assumption may be less accurate for different catalyst ligands, the difference is expected to be within the error of the model.

In one or more embodiments, multi-dimensional features can be used to build the initial machine learning model. A mix of physical features and molecular features were tested, and the physical features corresponded more to reaction conditions, including catalyst loading, co-catalyst loading, co-catalyst type, ethylene pressure, reaction temperature, and time. Table 1 below shows descriptions of two-dimensional molecular features used in the K value predictions:

TABLE 1
Descriptions of 2D Molecular Features Used in the Machine Learning Model:

| Features | Description |
|---|---|
| AMID_N | Averaged molecular ID on N atoms; considers general structure |
| | near nitrogen atoms |
| Xc-5dv | Valence 5^{th} order cluster Chi index; considers bonding and |
| | valence electrons |
| SlogP-VSA1, | Subdivided surface area descriptor based on atomic logP (i.e., |
| SlogP-VSA2 | octanol/water partition coefficient) and estimated accessible van |
| | der Waals surface area; SlogP-VSA1 considers atoms with |
| | higher estimated hydrophilicity than those of SlogP-VSA2 |
| SMR-VSA7 | Subdivided surface area descriptor based on atomic contribution |
| | to total polarizability (i.e., molar refractivity) of the ligand and |
| | estimated accessible van der Waals surface area |
| SdssC | Sum of E-state indexes for all C atoms in the ligand with one |
| SaaaC | double bond and two single bonds, and that for all C atoms with |
| | three aromatic bonds; The E-state index considers the |
| | electronegativity of an atom and its surrounding chemical |
| | environment |

The seven features of Table 1 were selected from more than 1500 2D features that were extracted for the 116 structures. The number of 2D features used was limited to only seven because redundant and unrelated features in the machine learning model will introduce noise and decrease its performance. A feature was removed from the model if it 1) had a normalized feature importance lower than 0.005; or 2) correlated well with other more important features.

In addition to reaction conditions and 2D features, a new set of features can be designed specifically for Fe oligomerization catalysts referred to as the connective steric factors (CSF). The CSF feature set may include fifteen individual features that quantify and describe the steric size of groups that extend beyond the base ligand framework. These new features are called length_Cn, width_Cn, depth_Cn (n=2, 3, 4, 5, or 6). After testing, only the length_C6 feature provided significant accuracy in the machine learning model.

To train regressors of the machine learning model, nine regression algorithms were tested, including random forest, least absolute shrinkage, and selection operator (LASSO), elastic-net, Gaussian process, ridge, Bayesian ridge, gradient-boosting, and support vector regression with both a linear and radial basis function kernel. To avoid overfitting the machine learning model, a random sampling was performed 150 times with the data set randomly split into 80% training and 20% testing sets each time.

Additionally, a graph neural network (GNN) model was built. GNNs use a graph representation of the molecule, where atoms are graph nodes and bonds are edges between nodes. Instead of the molecular features, one-hot encoded elements and bond orders were used as the initial properties for the nodes and edges. Through successive convolutions of adjacent nodes, information about the structure is shared to produce a set of weights. The weights are summed to give the predicted K value. The GNN model utilized six edge-conditioned convolution layers with 32 channels, as well as a global attention sum pool, which learns which node weights to sum during the training process. Reaction conditions were not included in the GNN model. The GNN model was subjected to the same cross validation methods as the other models.

The RMSE of all the regression algorithms ranged from 0.06 to 0.5 for the K values. The best performing model was random forest (RMSE=0.06). The random forest regressor is an ensemble (forest) of decision trees. Each tree is trained on a subset of the full training data set and, therefore, generates a slightly different prediction model. The final random forest model is the averaged results of all the decision trees. A random forest regressor is useful because it can generally handle outliers and unbalanced training data, and it is resistant to data overfitting. Other tested regressors showed similar performance, but they are slightly worse than the random forest (RMSE of ˜0.1). For Gaussian process regression, several kernels were tested. The rational quadratic kernel slightly outperformed the radial basis function kernels, which tended to overfit during hyperparameter optimization. The performance of support vector regression improved significantly when changing from a linear (RMSE=0.50) to a radial basis function kernel (RMSE=0.12). The GNN model performed well with an RMSE of 0.07.

Propagation (e.g., migratory insertion) and termination (e.g., β-hydrogen transfer) transition-state energy calculations at the M06-L/def2-TZVP//M06-L/6-31G**[LANL2DZ for Fe] level give K(C₁₂/C₁₀) values of 0 for all three complexes I (L₁=Me, Et, iPr), predicting the absence of C-chain propagation during catalysis. In comparison, the experimentally measured K values for complexes I (L₁=Me, Et, iPr) range between 0.6 and 0.8 under varying reaction conditions. Single point DLPNO-CCSD(T) using DFT-optimized geometries with the RIJCOSX approximation at the def2-TZVP//def2-TZVP/C//def2/J levels also result in K values very close to 0. Therefore, both DFT and DLPNO-CCSD(T) are not accurate enough to model the oligomerization selectivity.

In the random forest model, the AMID_N is statistically the most important feature for predicting Fe catalyst K values, followed by SlogP_VSA2 and length_C6. The AMID_N is the average molecular ID of nitrogen atoms and characterizes molecular branching around the nitrogen atoms. SlogP_VSA2 pertains to the estimated surface area of relatively hydrophilic atoms. As described above, the length_C6 parameter describes the size of the ligand arm branching from the main ligand core surrounding the Fe metal center, which is a CSF feature. The relative importance of AMID_N and length_C6 suggests that the K value of catalysts is heavily influenced by the steric impact of ligand arms, as well as the general structure of the backbone. This interpretation demonstrates that chemical properties that control selectivity can be qualitatively identified through machine learning analysis.

Although the other molecular features are statistically less important, they are still very useful for the model and survived the feature selection process. These features either directly or indirectly describe the electronic nature of the ligand scaffold. The SdssC parameter, which sums the E-states of carbons with a double bond and two single bonds, is indicative of the general ligand scaffold. For ligands with two imines or an imine and a ketone, the value of this parameter is typically around 2-3. If there is just one imine (e.g., phenanthroline-imine ligands), the value is typically around 1-1.5. The closely related SaaaC parameter (i.e., sum of E-states on carbons with three aromatic bonds) can also be useful for classifying ligands by backbone. Carbons with three aromatic bonds are only present in phenanthroline and α-diimine ligands in the training set. The SlogP-VSA1 parameter is the estimated surface area of very hydrophilic atoms. This parameter provides an indirect measure of aromatic heteroatoms. Similarly, SMR-VSA7 estimates the surface area of relatively polarizable atoms. For the machine learning training set, these are primarily aryl halides, atoms coordinated to the iron (which have a positive formal charge in the input structures), and aromatic carbons bonded to aliphatic carbons. Even though the physical features (reaction conditions) have lower importance than molecular features, the machine learning model can predict the changes in K value with respect to different reaction conditions.

The efficacy of the random forest model was determined where either only physical or only molecular features were used. When only the six physical features were used, the random forest model was only able to predict K(C₁₂/C₁₀) values with moderate to poor accuracy (test set gave an averaged R²=0.42 over 150 random samplings (see SI). Despite the poor model performance, feature importance did reveal that the most important physical features for predicting K values are the ethylene pressure and then catalyst loading. However, both physical features show little importance in the random forest model when physical and molecular features are included.

In contrast to the random forest model with only physical features, a random forest model with only molecular features provides almost the same accuracy as the model with all 14 features. The random forest model predicted K(C₁₂/C₁₀) values with an averaged R²=0.74. Analysis of the feature importance suggests that, like the physical and chemical model, the AMID_N, SlogP_VSA2, and length_C6 features are most important. Overall, the comparison of these models with only physical and only chemical features (molecular features and CSF features) indicates that the selectivity for Fe ethylene oligomerization catalysis is governed and dominated by the ligand impacting the steric and electronics of the Fe metal center and the transition states for propagation versus termination. Therefore, further examination of ligand steric and electronic effects was conducted using the optimized machine learning model with only chemical features.

To demonstrate that this random forest model provides prediction of key steric effects, the model was used to examine the effect of methyl (-Me) versus ethyl (-Et) versus isopropyl (-iPr) groups in the aryl ortho position of ligand arms. This is important because it is extremely difficult, if not impossible, for DFT calculations to predict (quantitatively or qualitatively) this ligand effect. Within the experimental data set, fifteen sets of K values, corresponding to eleven groups of catalysts, were considered. Each group of catalysts consists of three catalysts that have the same ligand backbone but have different substitutions on the phenyl-imine arm. The machine learning model can capture relationships where the K value increases with increasing group bulkiness, where the K value has an inverse relationship group bulkiness, and where there is no specific pattern.

In one or more embodiments, chemical structures including an Fe metal center and three monodentate ligands, a bidentate ligand and a monodentate ligand, or a tridentate ligand can be converted to a simplified molecular-input line-entry system (SMILES) string representing the chemical structure as American Standard Code for Information Interchange (ASCII) strings. Chemical features (molecular features and CSF features) of the chemical structure can be generated based on a

$$K ( \frac{{C}_{12}}{{C}_{10}} )$$

value measuring selectivity for propagation versus termination during oligomerization catalysis for the chemical structure. From the chemical features, a subset of them can be selected for training the machine learning model to identify ligand structures, with the subset including CSF features. The subset and first

$$K ( \frac{{C}_{12}}{{C}_{10}} )$$

value's for ethylene oligomerization catalyst structures can train the model to predict second

$$K ( \frac{{C}_{12}}{{C}_{10}} )$$

values based on sets of the chemical features for respective iron ethylene oligomerization catalyst structures.

Table 2 below shows example machine learning predicted K values using the machine learning model:

TABLE 2
Machine Learning Predicted K Values:

| | R₁ | R₂ | R₃ | # | K pred. |
|---|---|---|---|---|---|
| 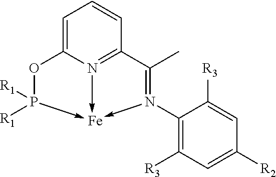 | Ph Ph Ph Ph tBu tBu tBu tBu iPr iPr iPr | H H Me Me H H Me Me H H Me | Me H Me H Me H Me H Me H Me | 17 18 19 20 21 22 23 24 25 26 27 | 0.57 0.57 0.57 0.57 0.69 0.62 0.69 0.63 0.69 0.62 0.69 |
| | iPr | Me | H | 28 | 0.63 |
| | CyHex | H | Me | 29 | 0.72 |
| | CyHex | H | H | 30 | 0.72 |
| | CyHex | Me | Me | 31 | 0.72 |
| | CyHex | Me | H | 32 | 0.72 |

The K value prediction workflow can include designing a chemical structure input. The general formula of the structure can be L₃FeCl₂ (e.g., monodentate ligands), LaFeCl₂ (e.g., one bidentate and one monodentate ligand), or LFeCl₂(e.g., one tridentate ligand). Two methods can be used: (1) a 2D molecular connectivity map, or (2) a 3D chemical structure based on spatial X, Y, Z atomic coordinates. The structure can be converted to a computer-readable string (e.g., SMILES, representing ASCII strings). The computer-readable strings can be used to predict the features above (e.g., Table 1 and the CSF factors). The potential catalyst structure can be imported into software to generate the features of Table 1 in preparation for execution of the machine learning model. The features in Table 1 can serve to identify ligand structures with information regarding atomic connectivity, sterics, electronic properties, topologies, hydrophilicity, and polarizability. The eighth feature is the CSF factors with fifteen sub-features to quantify and describe ligand arms combined with characteristic steric size. The features can be imported into the machine learning model to generate K value predictions.

The above descriptions are for purposes of illustration and are not meant to be limiting. Numerous other examples, configurations, processes, etc., may exist, some of which are described in greater detail below. Example embodiments will now be described with reference to the accompanying figures.

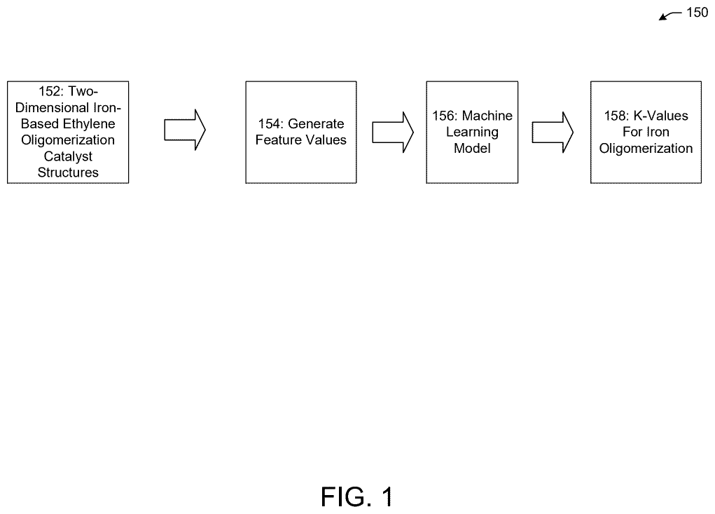

FIG. 1 is an exemplary process 150 for machine learning for iron-based ethylene oligomerization catalyst K value prediction in accordance with one embodiment.

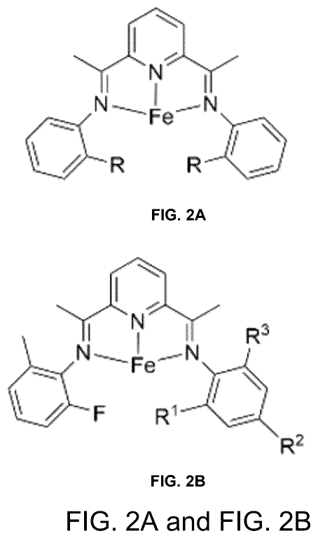

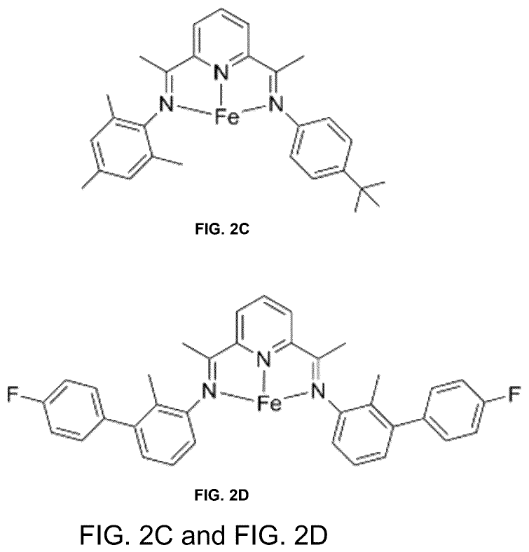

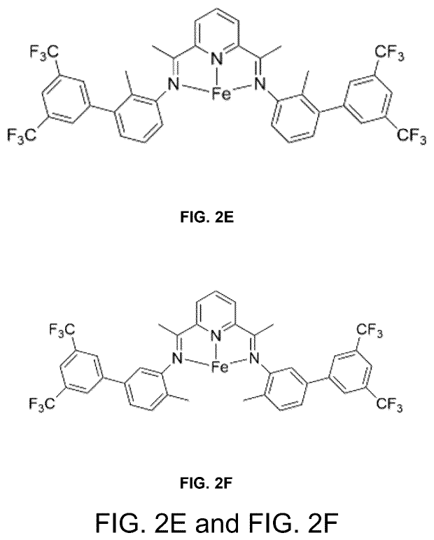

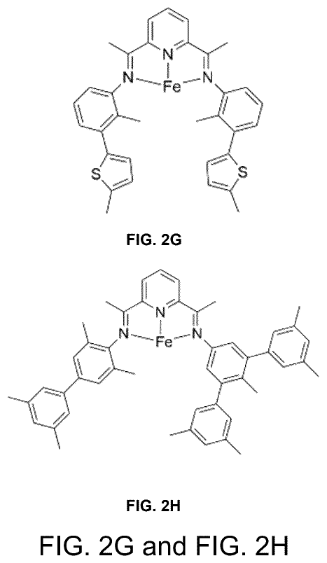

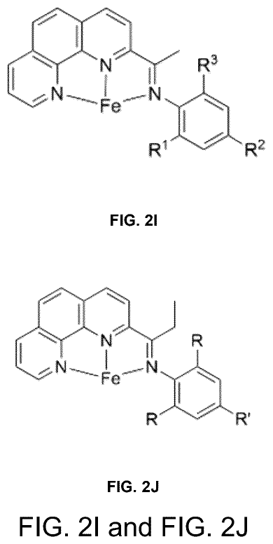

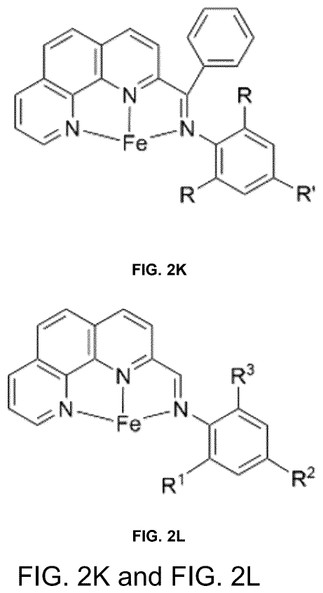

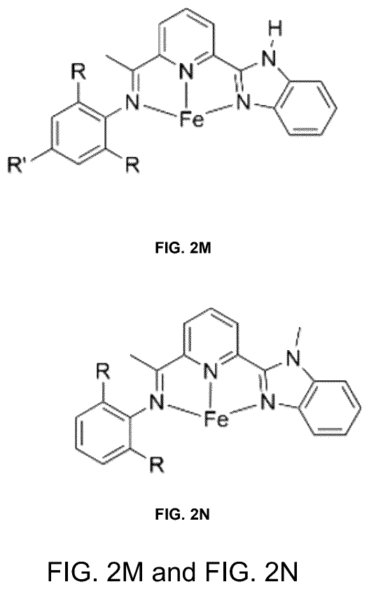

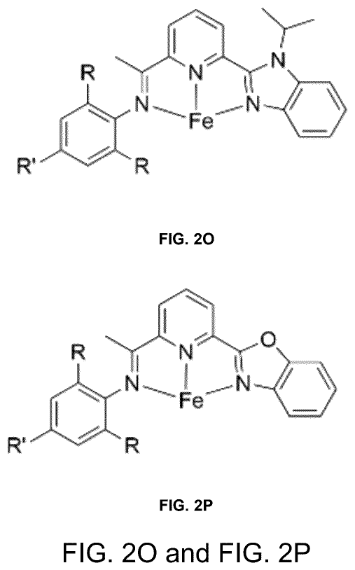

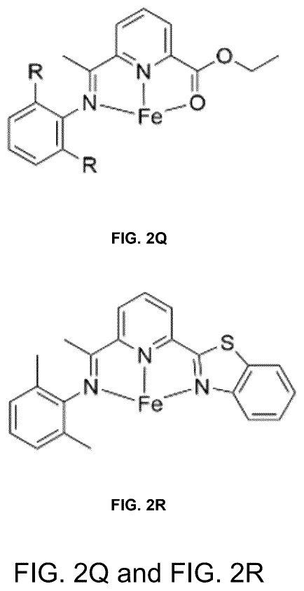

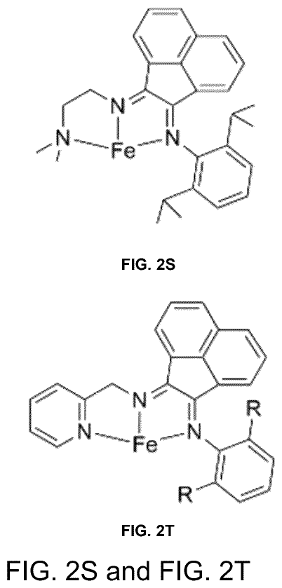

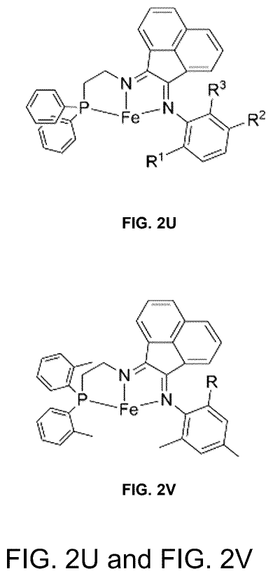

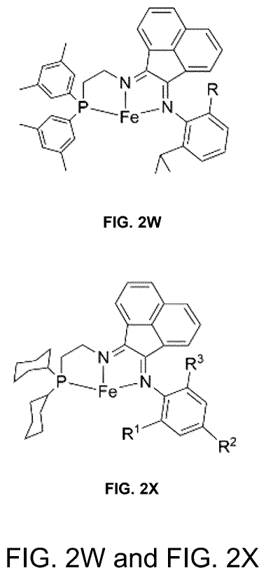

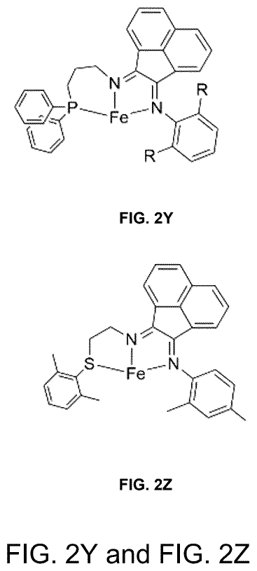

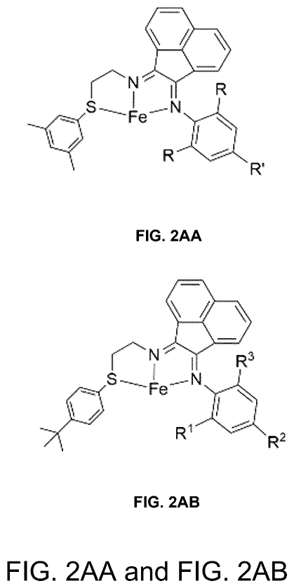

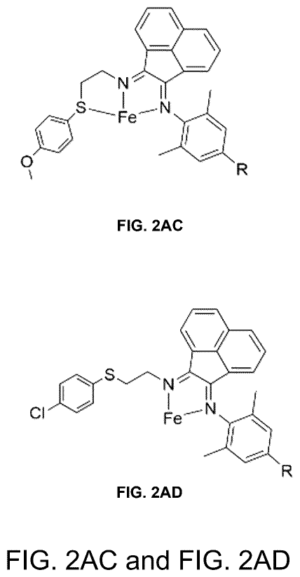

Referring to FIG. 1, process 150 may include step 152 in which 2D Fe-based ethylene oligomerization catalyst structures (e.g., shown in FIGS. 2A-2AD) stored in computer-readable format (e.g., the Simplified Molecular-Input Line-Entry System (SMILES) format)) are provided for feature analysis. At step 154, feature values may be generated for the 2D Fe-based ethylene oligomerization catalyst. At step 156, the features may be input to a machine learning model, which may evaluate the features and predict K values for the Fe-based ethylene oligomerization catalyst structures (e.g., predicted K(C₁₂/C₁₀) value for each structure; K(C₁₄/C₁₂) values may be converted to K(C₁₂/C₁₀) values when applicable).

To initially generate the machine learning model, more than 1500 different 2D molecular features were generated at step 154 for the machine learning data set. To increase model efficiency, redundant and unrelated features in the machine learning model were removed following four steps: (1) A feature was removed if a non-numerical value was generated for any structure within the machine learning data set. This step removed around 400 features. (2) A feature was removed if its corresponding normalized feature importance determined from random forest model was lower than 0.005. An additional 1150 features were removed based on this criterion. (3) A feature was removed if it well-correlated with another feature and if the former feature had lower importance than the latter. Two features were considered well-correlated with each other if the standard correlation coefficient was higher than 0.83. (4) A subset of the 2D features was kept in the ML model after steps (1)-(3). Then, selected combinations of features were used to increase model accuracy based on computed averaged RMSE value. Additional features were removed, most of which had normalized feature importance lower than 0.015.

With the exception of the graph neural network (GNN), machine learning models used for the machine learning model at step 156 may be trained against experimental K values and selected features. The GNN model may be trained using a different library.

To train and test the machine learning model at step 156, the data set was first randomly divided into training (e.g., 80% of the data set) and testing (e.g., 20% of the data set) data sets.

Ten machine learning algorithms were trained using the training set; the accuracy of each algorithm was then evaluated using the testing set. This process was repeated 150 times, each with randomly chosen training and testing sets to avoid overfitting an algorithm to the data set. Over-fitted machine learning models can give high accuracy for the given testing set but often generate poor prediction for new structures outside of the original data set. Feature importance is generated after model training and is used for the feature selection of the features of step 154. The selected features of step 154 are shown in Table 1 above.

The training data set included experimentally determined K values, physical features (reaction conditions for the experimentally determined K values), molecular features, and CSF features for the catalyst structures. Regarding K values and physical features, the catalyst structures of step 152 each had one or more K values corresponding to one or more sets of reaction conditions (e.g., catalyst loading (including co-catalyst loading), cocatalyst identity, ethylene pressure, time, and reaction temperature) for each catalyst structure. Regarding molecular features, the seven features listed in Table 1 were selected for each catalyst structure in the training data set from among more than 1500 2D features tested as described above. Regarding corrective steric factors, fifteen CSF features were selected: length_Cn, width_Cn, depth_Cn (n=2, 3, 4, 5, or 6) for each catalyst structure in the training data set. Nine regression algorithms were trained for the machine learning model at step 156, including random forest, least absolute shrinkage, and selection operator (LASSO), elastic-net, Gaussian process, ridge, Bayesian ridge, gradient-boosting, and support vector regression with both a linear and radial basis function kernel.

The testing data set (the 20% of the overall data set of iron ethylene oligomerization catalyst structures that had known K values and that was not used for training in order to assess the accuracy of the trained algorithms), was used to assess the accuracy of the algorithms by comparing the predicted K value for the structures in the testing data set with the known K values of the structures in the testing data set. Random forest performed the best, with a RMSE for predicting the K values 158 using the selected features of step 154 compared to the experimentally determined K values for the structures in the testing data set. The random forest regressor is an ensemble (forest) of decision trees. Each tree is trained on a subset of the full training data set and, therefore, generates a slightly different prediction model. The final random forest model is the averaged results of all the decision trees. A random forest regressor is useful because it can generally handle outliers and unbalanced training data, and it is resistant to data overfitting.

It was found that the molecular feature of AMID_N is statistically the most important feature for predicting Fe catalyst K values, followed by SlogP_VSA2 and length_C6. The AMID_N is the average molecular ID of nitrogen atoms and characterizes molecular branching around the nitrogen atoms. SlogP_VSA2 pertains to the estimated surface area of relatively hydrophilic atoms. As described above, the length_C6 parameter describes the size of ligand arm branching from the main ligand core surrounding the Fe metal center, which is a CSF feature. The relative importance of AMID_N and length_C6 suggests that the K value of catalysts is heavily influenced by the steric impact of the ligand arm(s), as well as the general structure of the backbone. This interpretation demonstrates that chemical properties that control selectivity can be qualitatively identified through machine learning analysis.

To demonstrate that this random forest model provides prediction of key steric effects, the machine learning model at step 156 was used to examine the effect of methyl (-Me) versus ethyl (-Et) versus isopropyl (-iPr) groups in the aryl ortho position of ligand arms. This is important because it is extremely difficult, if not impossible, for DFT calculations to predict (quantitatively or qualitatively) this ligand effect.

To begin to validate the machine learning model at step 156, a prediction was made for a Fe complex that had not previously been tested for olefin oligomerization selectivity (a new iron ethylene oligomerization catalyst structure). This new catalyst is shown below:

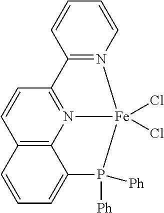

The new catalyst above features a pyridylquinolinylphosphine (PQP) type ligand structure. The new catalyst structure was input into the random forest machine learning model at step 152 in FIG. 1. Feature values were generated at step 154, and at step 156, the random forest machine learning model predicted a K value for this (PQP)Fe catalyst of 0.58. Subsequent experimental measurement of the K(C₁₂/C₁₀) value was 0.52. Implicitly, the experimental K value was determined for the same reaction conditions for which the predicted K value was predicted. This validation demonstrates the potential quantitative utility of this machine learning model. However, like any machine learning model caution should be used when designing and predicting new catalysts that are outside of the training data. This (PQP)Fe catalyst fits within the training data because it has both a phosphine and pyridine type direct ligation to the Fe metal center. This experimental validation shows the utility of developing a specific machine learning to enable catalyst development.

FIG. 2A illustrates an example iron-based ethylene oligomerization catalyst used in a data set to train and test the machine learning model of FIG. 1 in accordance with one embodiment.

The complexes for the catalyst in FIG. 2A include: (1) R=Me, (2) R=Et, (3) R=jPr.

FIG. 2B illustrates an example iron-based ethylene oligomerization catalyst used in a data set to train and test the machine learning model of FIG. 1 in accordance with one embodiment.

The complexes for the catalyst in FIG. 2B include: (4) R¹=F, R²=H, R³=Me; (5) R¹=H, R²=H, R³=Me; (6) R¹=F, R²=Me, R³=Me.

FIG. 2C illustrates an example iron-based ethylene oligomerization catalyst 7 used in a data set to train and test the machine learning model of FIG. 1.

FIG. 2D illustrates an example iron-based ethylene oligomerization catalyst used in a data set to train and test the machine learning model of FIG. 1.

FIG. 2E illustrates an example iron-based ethylene oligomerization catalyst used in a data set to train and test the machine learning model of FIG. 1.

FIG. 2F illustrates an example iron-based ethylene oligomerization catalyst used in a data set to train and test the machine learning model of FIG. 1.

FIG. 2G illustrates an example iron-based ethylene oligomerization catalyst used in a data set to train and test the machine learning model of FIG. 1.

FIG. 2H illustrates an example iron-based ethylene oligomerization catalyst used in a data set to train and test the machine learning model of FIG. 1.

FIG. 21 illustrates an example iron-based ethylene oligomerization catalyst used in a data set to train and test the machine learning model of FIG. 1.

The complexes for the catalyst in FIG. 21 include: (13) R¹=Me, R²=H, R³=Me; (14) R¹=Et, R²=H, R³=Et; (15) R¹=iPR, R²=H, R³=iPr; (16) R¹=F, R²=H, R³=F; (17) R¹=Cl, R²=H, R³=Cl; (18) R¹=Br, R²=H, R³=Br; (19) R¹=Me, R²=Me, R³=Me; (20) R¹=Me, R²=Br, R³=Me; (21) R¹=Br, R²=Me, R³=Br; (22) R¹=Br, R²=Cl, R³=Br; (23) R¹=Br, R²=Br, R³=Br; (24) R¹=Me, R²=H, R³=H; (25) R¹=Me, R²=Me, R³=H; (26) R¹=Et, R²=H, R³=H; (27) R¹=iPR, R²=H, R³=H; (28) R¹=Me, R²=H, R³=Et; (29) R¹=Br, R²=H, R³=H; (30) R¹=Br, R²=Me, R³=H; (31) R¹=Br, R²=F, R³=H; (32) R¹=Br, R²=Br, R³=H; (33) R¹=F, R²=F, R³=F; (34) R¹=Cl, R²=Cl, R³=Cl; (35) R¹=Br, R²=F, R³=F; (36) R¹=Br, R²=F, R³=Cl; (37) R¹=Br, R²=Cl, R³=F.

FIG. 2J illustrates an example iron-based ethylene oligomerization catalyst used in a data set to train and test the machine learning model of FIG. 1.

The complexes for the catalyst in FIG. 2J include: (38) R=Me, R′=H; (39) R=Et, R′=H; (40) R=iPr, R′=H; (41) R=F, R′=H; (42) R=Cl, R′=H; (43) R=Me, R′=Me; (44) R=Et, R′=Me.

FIG. 2K illustrates an example iron-based ethylene oligomerization catalyst used in a data set to train and test the machine learning model of FIG. 1.

The complexes for the catalyst in FIG. 2K include: (45) R=Me, R′=H; (46) R=Et, R′=H; (47) R=iPr, R′=H; (48) R=Me, R′=Me; (49) R=Me, R′=Br.

FIG. 2L illustrates an example iron-based ethylene oligomerization catalyst used in a data set to train and test the machine learning model of FIG. 1.

The complexes for the catalyst in FIG. 2L include: (50) R¹=Me, R²=H, R³=Me; (51) R¹=Et, R²=H, R³=Et; (52) R¹=iPr, R²=H, R³=iPr; (53) R¹=F, R²=H, R³=F; (54) R¹=Cl, R²=H, R³=Cl; (55) R¹=Br, R²=H, R³=Br; (56) R¹=Me, R²=H, R³=H; (57) R¹=Me, R²=Me, R³=Me; (58) R¹=Me, R²=Br, R³=Me.

FIG. 2M illustrates an example iron-based ethylene oligomerization catalyst used in a data set to train and test the machine learning model of FIG. 1.

The complexes for the catalyst in FIG. 2M include: (59) R=Me, R′=H; (60) R=Et, R′=H; (61) R=iPr, R′=H; (62) R=Cl, R′=H; (63) R=Br, R′=H; (64) R=Me, R′=Me.

FIG. 2N illustrates an example iron-based ethylene oligomerization catalyst used in a data set to train and test the machine learning model of FIG. 1.

The complexes for the catalyst in FIG. 2N include: (65) R=Me; (66) R=Et, (67) R=iPr, (68) R=C1, (69) R=Br.

FIG. 2O illustrates an example iron-based ethylene oligomerization catalyst used in a data set to train and test the machine learning model of FIG. 1.

The complexes for the catalyst in FIG. 2O include: (70) R=Me, R′=H; (71) R=Et, R′=H; (72) R=iPr, R′=H; (73) R=Me, R′=Me; (74) R=Me, R′=Br.

FIG. 2P illustrates an example iron-based ethylene oligomerization catalyst used in a data set to train and test the machine learning model of FIG. 1.

The complexes for the catalyst in FIG. 2P include: (75) R=Me, R′=H; (76) R=Et, R′=H; (77) R=iPr, R′=H; (78) R=Cl, R′=H; (79) R=Br, R′=H; (80) R=Me, R′=Me; (81) R=Me, R′=Br.

FIG. 2Q illustrates an example iron-based ethylene oligomerization catalyst used in a data set to train and test the machine learning model of FIG. 1.

The complexes for the catalyst in FIG. 2Q include: (82) R=Me; (83) R=Et; (84)R=iPr; (85) R=F; (86) R=Cl; (87) R=Br.

FIG. 2R illustrates an example iron-based ethylene oligomerization catalyst used in a data set to train and test the machine learning model of FIG. 1.

FIG. 2S illustrates an example iron-based ethylene oligomerization catalyst used in a data set to train and test the machine learning model of FIG. 1.

FIG. 2T illustrates an example iron-based ethylene oligomerization catalyst used in a data set to train and test the machine learning model of FIG. 1.

The complexes for the catalyst in FIG. 2T include: (90) R=Me; (91) R=iPr.

FIG. 2U illustrates an example iron-based ethylene oligomerization catalyst used in a data set to train and test the machine learning model of FIG. 1.

The complexes for the catalyst in FIG. 2U include: (92) R¹=Me, R²=H, R³=Me; (93) R¹=iPr, R²=H, R³=Me; (94) R¹=iPr, R²=H, R³=iPr; (95) R¹=tBu, R²=tBu, R³=H.

FIG. 2V illustrates an example iron-based ethylene oligomerization catalyst used in a data set to train and test the machine learning model of FIG. 1.

The complexes for the catalyst in FIG. 2V include: (96) R=Me; (97) R=He.

FIG. 2W illustrates an example iron-based ethylene oligomerization catalyst used in a data set to train and test the machine learning model of FIG. 1.

The complexes for the catalyst in FIG. 2W include: (98) R=Me; (99) R=iPr.

FIG. 2X illustrates an example iron-based ethylene oligomerization catalyst used in a data set to train and test the machine learning model of FIG. 1.

The complexes for the catalyst in FIG. 2X include: (100) R¹=Me, R²=Me, R³=H; (101) R¹=Me, R²=Me, R³=Me; (102) R¹=iPr, R²=H, R³=iPr.

FIG. 2Y illustrates an example iron-based ethylene oligomerization catalyst used in a data set to train and test the machine learning model of FIG. 1.

The complexes for the catalyst in FIG. 2Y include: (103) R¹=Me; (104) R¹=iPr.

FIG. 2Z illustrates an example iron-based ethylene oligomerization catalyst used in a data set to train and test the machine learning model of FIG. 1.

FIG. 2AA illustrates an example iron-based ethylene oligomerization catalyst used in a data set to train and test the machine learning model of FIG. 1.

The complexes for the catalyst in FIG. 2AA include: (106) R=Me, R′=Me; (107) R=iPr, R′=H.

FIG. 2AB illustrates an example iron-based ethylene oligomerization catalyst used in a data set to train and test the machine learning model of FIG. 1.

The complexes for the catalyst in FIG. 2AB include: (108) R¹=Me, R²=H, R³=Me; (109) R¹=Me, R²=Me, R³=H; (110) R¹=Me, R²=Me, R³=Me; (112) R¹=Me, R²=Br, R³=Me.

FIG. 2AC illustrates an example iron-based ethylene oligomerization catalyst used in a data set to train and test the machine learning model of FIG. 1.

The complexes for the catalyst in FIG. 2AC include: (113) R=H; (114) R=Me.

FIG. 2AD illustrates an example iron-based ethylene oligomerization catalyst used in a data set to train and test the machine learning model of FIG. 1.

The complexes for the catalyst in FIG. 2AC include: (115) R=H; (116) R=Me.

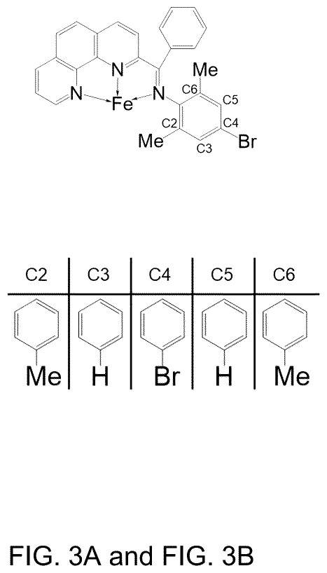

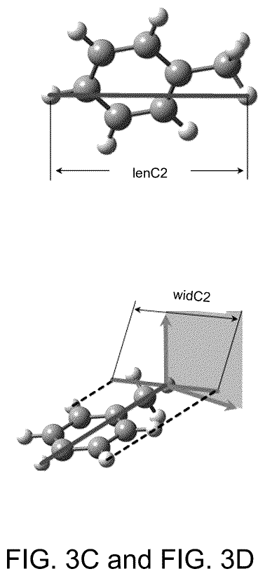

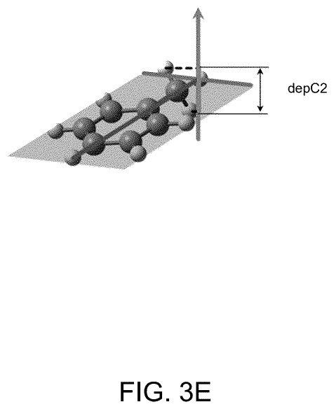

FIG. 3A illustrates an example complex of the iron-based ethylene oligomerization catalyst of FIG. 2K in accordance with one embodiment.

FIG. 3B illustrates example molecules for the C positions of the phenyl ring of the complex of FIG. 3A in accordance with one embodiment.

FIG. 3C illustrates an example length of a methylbenzene molecule of the complex of FIG. 3A in accordance with one embodiment.

FIG. 3D illustrates an example width of a methylbenzene molecule of the complex of FIG. 3A in accordance with one embodiment.

FIG. 3E illustrates an example depth of a methylbenzene molecule of the complex of FIG. 3A in accordance with one embodiment.

The determination of length_Cₙ, width_Cₙ, depth_Cₙ (n=2, 3, 4, 5, or 6) for a given Fe catalyst is described here using examples for the iron-based ethylene oligomerization catalyst of FIG. 2K.

The iron-based ethylene oligomerization catalyst of FIG. 2K, as shown in FIG. 3A, has one ligand arm, which is constituted of one phenyl ring. Because a methyl (-Me) group occupies the C2 position of the substituted phenyl ring, features length_C2, width_C2, and depth_C2 may be defined to be respectively the length, width, and depth of a methylbenzene molecule (e.g., as shown in FIGS. 3B and 3C). Similarly, length_C4, width_C4, and depth_C4 are defined as respectively the length, width, and depth of a bromobenzene molecule because a bromo group is at the C4 position of phenyl arm. Because the C6 position is also occupied by a methyl group as shown in FIG. 3A, features length_C6, width_C6, and depth_C6 are the same as length_C2, width_C2, and depth_C2, respectively. For C positions (C3 and C5) where there are no substitution groups, the dimension of a benzene molecule is used.

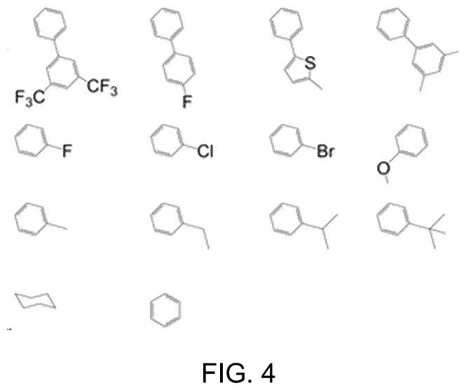

To determine the bulkiness for the Cₙ position (n=2, 3, 4, 5, or 6) of a ligand arm, the dimensions of a substituted benzene molecule may be used instead of the substitute group alone. This is done to ensure the measured length_Cn always follows the general direction of the phenyl-substitute bond as shown in FIG. 3A. To measure the dimensions of a substituted benzene molecule, geometry optimizations were performed for the molecule (e.g., using a semiempirical method). Among all the Fe catalysts contained in the machine learning data set, there are different substitution groups appearing on the phenyl ring of ligand arms (e.g., shown in FIG. 4). Their corresponding dimensions are predetermined and tabulated for the easy calculation of length_Cn, width_Cn, depth_Cn (n=2, 3, 4, 5, 6) for all the Fe catalysts.

FIG. 4 illustrates example molecules for generating a characteristic size of substitution groups on polydentate ligand arms of the iron-based ethylene oligomerization catalysts of FIGS. 2A-2AD in accordance with one embodiment.

As noted above, the molecules of FIG. 4 represent the substitution groups appearing on the phenyl ring of ligand arms.

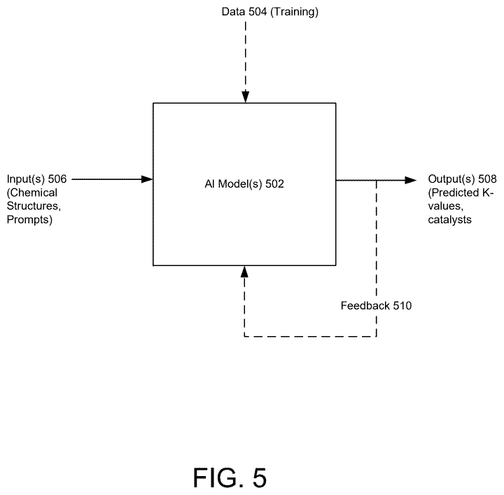

FIG. 5 is a schematic diagram of machine learning for the process 150 of FIG. 1 in accordance with one embodiment.

Referring to FIG. 5, one or more artificial intelligence (AI) models 502 (e.g., including the machine learning model at step 156 of FIG. 1) can be used to generate ethylene K value predictions based on inputs 506 (e.g., chemical structures with an Fe metal center and three monodentate ligands, a bidentate ligand and a monodentate ligand, or a tridentate ligand; AI prompts) and optional data 504 (e.g., training data). The one or more AI models 502 can predict outputs 508 (e.g., predicted K values), and optional feedback 510 (e.g., indicating accuracy of the predicted K values) can be fed back into the one or more AI models 502 to refine criteria used to predict the K values.

In one or more embodiments, multi-dimensional features can be used to build the initial machine learning model. A mix of physical features and molecular features were tested (e.g., as the data 504), and the physical features corresponded more to reaction conditions, including catalyst loading, co-catalyst loading, co-catalyst type, ethylene pressure, reaction temperature, and time. Table 1 above shows descriptions of two-dimensional molecular features used in the K value predictions.

The seven features of Table 1 were selected from more than 1500 2D features that were extracted for the 116 structures. The number of used 2D features was limited to only seven because redundant and unrelated features in the one or more AI models 502 will introduce noise and decrease its performance. A feature was removed from the one or more AI models 502 if it 1) had a normalized feature importance lower than 0.005; or 2) correlated well with other more important features.

In addition to reaction conditions and 2D features, a new set of features can be designed specifically for Fe oligomerization catalysts referred to as the connective steric factors (CSF). The CSF feature set may include fifteen individual features that quantify and describe the steric size of groups that extend beyond the base ligand framework. These new features are called length_Cn, width_Cn, depth_Cn (n=2, 3, 4, 5, or 6). After testing, only the length_C6 feature provided significant accuracy in the one or more AI models 502.

To train regressors of the one or more AI models 502, nine regression algorithms were tested, including random forest, least absolute shrinkage, and selection operator (LASSO), elastic-net, Gaussian process, ridge, Bayesian ridge, gradient-boosting, and support vector regression with both a linear and radial basis function kernel. To avoid overfitting the machine learning model, a random sampling was performed 150 times with the data set randomly split into 80% training and 20% testing sets each time.

Additionally, a graph neural network (GNN) model was built for the one or more AI models 502. GNNs use a graph representation of the molecule, where atoms are graph nodes and bonds are edges between nodes. Instead of the molecular features, one-hot encoded elements and bond orders were used as the initial properties for the nodes and edges. Through successive convolutions of adjacent nodes, information about the structure is shared to produce a set of weights. The weights are summed to give the predicted K value. The GNN model utilized six edge-conditioned convolution layers with 32 channels, as well as a global attention sum pool, which learns which node weights to sum during the training process. Reaction conditions were not included in the GNN model. The GNN model was subjected to the same cross validation methods as the other models.

The RMSE of all the regression algorithms ranged from 0.06 to 0.5 for the K values. The best performing model was random forest (RMSE=0.06). The random forest regressor is an ensemble (forest) of decision trees. Each tree is trained on a subset of the full training data set and, therefore, generates a slightly different prediction model. The final random forest model is the averaged results of all the decision trees. A random forest regressor is useful because it can generally handle outliers and unbalanced training data, and it is resistant to data overfitting. Other tested regressors showed similar performance, but they are slightly worse than the random forest (RMSE of ˜0.1). For Gaussian process regression, several kernels were tested. The rational quadratic kernel slightly outperformed the radial basis function kernels, which tended to overfit during hyperparameter optimization. The performance of support vector regression improved significantly when changing from a linear (RMSE=0.50) to a radial basis function kernel (RMSE=0.12). The GNN model performed well with an RMSE of 0.07.

Propagation (e.g., migratory insertion) and termination (e.g., β-hydrogen transfer) transition-state energy calculations at the M06-L/def2-TZVP//M06-L/6-31G**[LANL2DZ for Fe] level give K(C₁₂/C₁₀) values of 0 for all three complexes I (L1=Me, Et, iPr), predicting the absence of C-chain propagation during catalysis. In comparison, the experimentally measured K values for complexes I (L1=Me, Et, iPr) range between 0.6 and 0.8 under varying reaction conditions. Single point DLPNO-CCSD(T) using DFT-optimized geometries with the RIJCOSX approximation at the def2-TZVP//def2-TZVP/C//def2/J level also results in K values very close to 0. Therefore, both DFT and DLPNO-CCSD(T) are not accurate enough to model the oligomerization selectivity.

In the random forest model, the AMID_N is statistically the most important feature for predicting Fe catalyst K values, followed by SlogP_VSA2 and length_C6. The AMID_N is the average molecular ID of nitrogen atoms and characterizes molecular branching around the nitrogen atoms. SlogP_VSA2 pertains to the estimated surface area of relatively hydrophilic atoms. As described above, the length_C6 parameter describes the size of ligand arm branching from the main ligand core surrounding the Fe metal center, which is a CSF feature. The relative importance of AMID_N and length_C6 suggests that the K value of catalysts is heavily influenced by the steric impact of the ligand arm(s), as well as the general structure of the backbone. This interpretation demonstrates that chemical properties that control selectivity can be qualitatively identified through machine learning analysis.

Although the other molecular features are statistically less important, they are still very useful for the model and survived the feature selection process. These features either directly or indirectly describe the electronic nature of the ligand scaffold. The SdssC parameter, which sums the E-states of carbons with a double bond and two single bonds, is indicative of the general ligand scaffold. For ligands with two imines or an imine and a ketone, the value of this parameter is typically around 2-3. If there is just one imine (e.g., phenanthroline-imine ligands), the value is typically around 1-1.5. The closely related SaaaC parameter (i.e., sum of E-states on carbons with three aromatic bonds) can also be useful for classifying ligands by backbone. Carbons with three aromatic bonds are only present in phenanthroline and α-diimine ligands in the training set. The SlogP-VSA1 parameter is the estimated surface area of very hydrophilic atoms. This parameter provides an indirect measure of aromatic heteroatoms. Similarly, SMR-VSA7 estimates the surface area of relatively polarizable atoms. For the machine learning training set, these are primarily aryl halides, atoms coordinated to the iron (which have a positive formal charge in the input structures), and aromatic carbons bonded to aliphatic carbons. Even though the physical features (reaction conditions) have lower importance than molecular features, the one or more AI models 502 can predict the changes in K value with respect to different reaction conditions.

The efficacy of the random forest model was determined where either only physical or only molecular features were used. When only the six physical features were used, the random forest model was only able to predict K(C₁₂/C₁₀) values with moderate to poor accuracy (test set gave an average R²=0.42 over 150 random samplings (see SI)). Despite the poor model performance, feature importance did reveal that the most important physical features for predicting K value are the ethylene pressure and then catalyst loading. However, both physical features show little importance in the random forest model when physical and molecular features are included.

In contrast to the random forest model with only physical features, a random forest model with only molecular features provides almost the same accuracy as the model with all 14 features. The random forest model predicted K(C₁₂/C₁₀) values with an average R²=0.74. Analysis of the feature importance suggests that, like the physical and chemical model, the AMID_N, SlogP_VSA2, and length_C6 features are most important. Overall, the comparison of these models with only physical and only chemical features (molecular features and CSF features) indicates that the selectivity for Fe ethylene oligomerization catalysis is governed and dominated by the ligand impacting the steric and electronics of the Fe metal center and the transition states for propagation versus termination. Therefore, further examination of ligand steric and electronic effects was conducted using the optimized machine learning model with only chemical features.

To demonstrate that this random forest model provides prediction of key steric effects, the model was used to examine the effect of methyl (-Me) versus ethyl (-Et) versus isopropyl (-iPr) groups in the aryl ortho position of ligand arms. This is important because it is extremely difficult, if not impossible, for DFT calculations to predict (quantitatively or qualitatively) this ligand effect. Within the experimental data set, fifteen sets of K values, corresponding to eleven groups of catalysts, were considered. Each group of catalysts consists of three catalysts that have the same ligand backbone but have different substitutions on the phenyl-imine arm. The one or more AI models 502 can capture relationships where the K value increases with increasing group bulkiness, where the K value has an inverse relationship group bulkiness, and where there is no specific pattern.

In one or more embodiments, chemical structures including an Fe metal center and three monodentate ligands, a bidentate ligand and a monodentate ligand, or a tridentate ligand can be converted to a simplified molecular-input line-entry system (SMILES) string representing the chemical structure as American Standard Code for Information Interchange (ASCII) strings. Chemical features (molecular features and CSF features) of the chemical structure can be generated based on a

$$K ( \frac{{C}_{12}}{{C}_{10}} )$$

value measuring selectivity for propagation versus termination during oligomerization catalysis for the chemical structure. From the chemical features, a subset of them can be selected for training the one or more AI models 502 to identify ligand structures, with the subset including CSF features. The subset and first

$$K ( \frac{{C}_{12}}{{C}_{10}} )$$

values for ethylene oligomerization catalyst structures can train the one or more AI models 502 to predict second

$$K ( \frac{{C}_{12}}{{C}_{10}} )$$

values based on sets of the chemical features for respective iron ethylene oligomerization catalyst structures.

Table 2 above shows example machine learning predicted K values using the machine learning model.

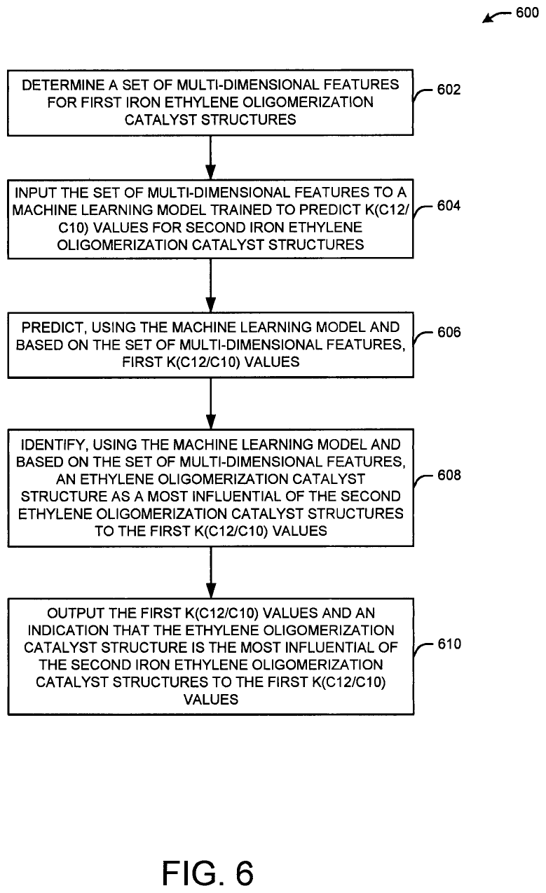

FIG. 6 is a flowchart illustrating a process 600 for machine learning for iron-based oligomerization of ethylene K value prediction in accordance with one embodiment.

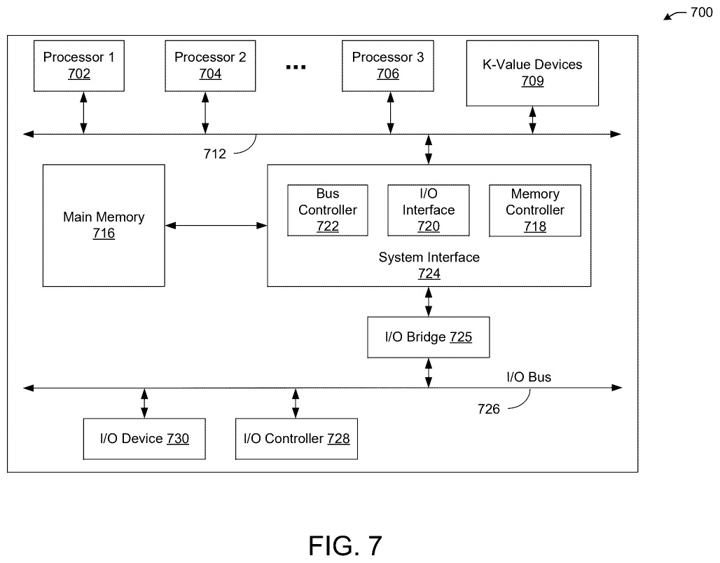

At block 602, a device (e.g., the system 700 of FIG. 7) can determine a set of multi-dimensional features for first iron ethylene oligomerization catalyst structures. Two methods may be used: (1) a 2D molecular connectivity map, or (2) a 3D chemical structure based on spatial X, Y, Z atomic coordinates. The structure can be converted to a computer-readable string (e.g., SMILES, representing ASCII strings). The computer-readable strings can be used to predict the features above (e.g., Table 1 and the CSF factors). The first iron ethylene oligomerization catalyst structures can include an Fe metal center coordinated with N, O, S, and/or P ligands. The first iron ethylene oligomerization catalyst structures can include an Fe metal center and pyridine-bisimine, α-diimine, phenanthroline, and iminopyridine ligands. The first iron ethylene oligomerization catalyst structures can include the following structures:

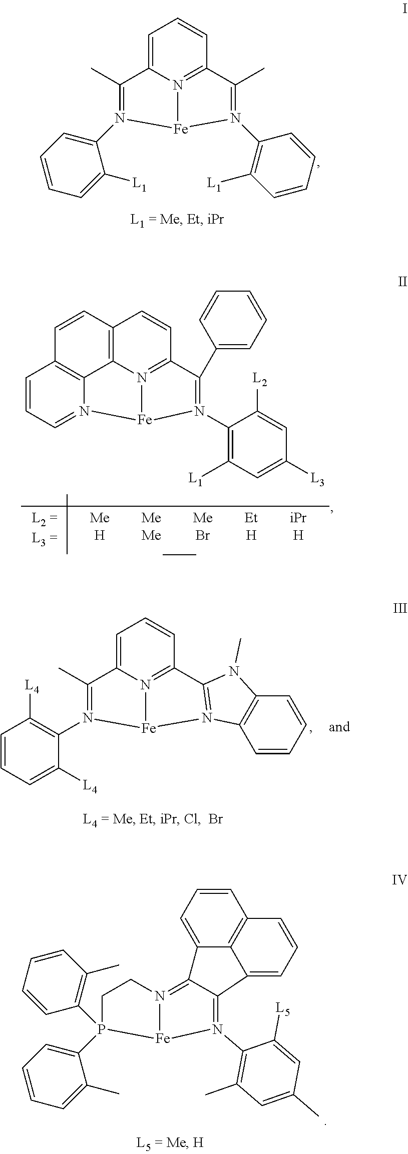

At block 604, the device can input the set of multi-dimensional features to a machine learning model (e.g., random forest) trained to predict

$$K ( \frac{{C}_{12}}{{C}_{10}} )$$

values for second iron ethylene oligomerization catalyst structures.

At block 606, the device can predict, using the machine learning model and based on the set of multi-dimensional features, first

$$K ( \frac{{C}_{12}}{{C}_{10}} )$$

values.

At block 608, the device an identify, using the machine learning model and based on the set of multi-dimensional features, an ethylene oligomerization catalyst structure as a most influential of the second ethylene oligomerization catalyst structures to the first

$$K ( \frac{{C}_{12}}{{C}_{10}} )$$

values. Identifying the ethylene oligomerization catalyst structure as the most influential of the second iron ethylene oligomerization catalyst structures to the first

$$K ( \frac{{C}_{12}}{{C}_{10}} )$$

values can be based on a proximity of the iron ethylene oligomerization catalyst structures to a subset of one or more second iron ethylene oligomerization catalyst structures of the second iron ethylene oligomerization catalyst structures.

At block 610, the device can output the first

$$K ( \frac{{C}_{12}}{{C}_{10}} )$$

values and an indication that the ethylene oligomerization catalyst structure is the most influential of the second iron ethylene oligomerization catalyst structures to the first

$$K ( \frac{{C}_{12}}{{C}_{10}} )$$

values.

It is understood that the above descriptions are for purposes of illustration and are not meant to be limiting.

FIG. 7 is a block diagram illustrating an example of a computing device or computer system 700 which can be used in implementing the embodiments of the components of the network disclosed above. For example, the computer system 700 of FIG. 7 can represent at least a portion of the components of FIG. 1 as discussed above. The computer system (“system”) includes one or more processors 702-706 and one or more K value devices 709 (e.g., capable of performing any operations described with respect to FIG. 1 and FIG. 6). Processors 702-706 can include one or more internal levels of cache (not shown) and a bus controller 722 or bus interface unit having direct interaction with the processor bus 712. Processor bus 712, also known as the host bus or the front side bus, can be used to couple the processors 702-706 with the system interface 724. System interface 724 can be connected to the processor bus 712 to interface other components of the system 700 with the processor bus 712. For example, system interface 724 can include a memory controller 718 for interfacing a main memory 716 with the processor bus 712. The main memory 716 typically includes one or more memory cards and a control circuit (not shown). System interface 724 can also include an input/output (I/O) interface 720 to interface one or more I/O bridges 725 or I/O devices with the processor bus 712. One or more I/O controllers and/or I/O devices can be connected with the I/O bus 726, such as I/O controller 728 and I/O device 730, as illustrated.

I/O device 730 can also include an input device (not shown), such as an alphanumeric input device, including alphanumeric and other keys for communicating information and/or command selections to the processors 702-706. Another type of user input device includes cursor control, such as a mouse, a trackball, or cursor direction keys for communicating direction information and command selections to the processors 702-706 and for controlling cursor movement on the display device.

System 700 can include a dynamic storage device, referred to as main memory 716, or a random-access memory (RAM) or other computer-readable devices coupled to the processor bus 712 for storing information and instructions to be executed by the processors 702-706. Main memory 716 also can be used for storing temporary variables or other intermediate information during execution of instructions by the processors 702-706. System 700 can include a read only memory (ROM) and/or other static storage device coupled to the processor bus 712 for storing static information and instructions for the processors 702-706. The system outlined in FIG. 7 is but one possible example of a computer system that can employ or be configured in accordance with aspects of the present disclosure.

According to one embodiment, the above techniques can be performed by computer system 700 in response to processor 704 executing one or more sequences of one or more instructions contained in main memory 716. These instructions can be read into main memory 716 from another machine-readable medium, such as a storage device. Execution of the sequences of instructions contained in main memory 716 can cause processors 702-706 to perform the process steps described herein. In alternative embodiments, circuitry can be used in place of or in combination with the software instructions. Thus, embodiments of the present disclosure can include both hardware and software components.

A machine-readable medium includes any mechanism for storing or transmitting information in a form (e.g., software, processing application) readable by a machine (e.g., a computer). Such media can take the form of, but is not limited to, non-volatile media and volatile media and may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, solid-state drives (SSDs), and the like. The one or more memory devices 706 may include volatile memory (e.g., dynamic random-access memory (DRAM), static random-access memory (SRAM), etc.) and/or non-volatile memory (e.g., read-only memory (ROM), flash memory, etc.).

Computer program products containing mechanisms to effectuate the systems and methods in accordance with the presently described technology can reside in main memory 716, which may be referred to as machine-readable media. It will be appreciated that machine-readable media can include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media can include a single medium or multiple media (e.g., a centralized or distributed database, and/or associated caches and servers) that store the one or more executable instructions or data structures.

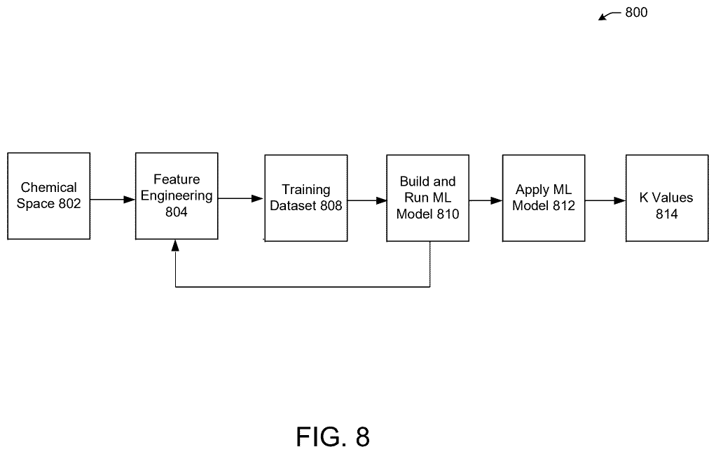

FIG. 8 is a flowchart illustrating a process 800 for developing new catalysts for the iron-based oligomerization in accordance with one embodiment.

The process 800 can include exploring a chemical space 802 and performing feature engineering 804 on features of the chemical space 802. From the features, a training dataset 808 can be developed for a ML model (e.g., the one or more AI models 502 of FIG. 5). Using the training dataset 808, the ML model can be built and run at step 810, and learnings can be fed back to the feature engineering 804 for updating of the features and the training dataset 808. By applying the ML model to new catalyst structures at step 812, K values 814 for new iron-based oligomerization catalyst structures can be predicted.

In one or more embodiments, the ML model can predict active versus inactive catalysts. For the ML model to predict inactive catalysts, the training dataset 808 can identify features that work and do not work for the catalysts. For example, regarding catalyst stability, if a catalyst falls apart immediately in an active environment, its productivity and K value are irrelevant. Regarding an insertion barrier, the barrier may be low, but if the complex is highly unstable, no oligomerization will occur. Conversely, a K value can be generated, but a high insertion barrier may not result in any products. Because the ML model can predict a K value for a catalyst that may not work, there can be thresholds used to interpret K values as irrelevant or impractical. For example, a K value of 0.0001 may indicate that characteristics of the catalyst are too limiting to be practical. In this manner, the ML model can be trained to identify impractical catalysts based on catalyst features and/or K values.

### Statements

The following disclosure Statements provide additional details of the methods, devices, and systems of this disclosure. Statements which are described as “comprising” certain components or steps, may also “consist essentially of” or “consist of” those components or steps, unless stated otherwise. Variations of these Statements will suggest themselves to those skilled in the art in light of the Detailed Description and Drawings which follows, and all such obvious variations are within the full intended scope of the appended claims.

Statement 1 can include a method for identifying catalyst structures comprising ligands for the iron-based oligomerization of ethylene, the method including: determining a set of multi-dimensional features for first iron ethylene oligomerization catalyst structures, wherein the set of multi-dimensional features comprises physical features corresponding to reaction conditions, comprises molecular features, and comprises connective steric factors; inputting the set of multi-dimensional features to a machine learning model trained to predict K(C₁₂/C₁₀) values measuring selectivity for propagation versus termination during oligomerization catalysis for second iron ethylene oligomerization catalyst structures based on the multi-dimensional features; predicting, using the machine learning model and based on the set of multi-dimensional features, first K(C₁₂/C₁₀) values; identifying, using the machine learning model and based on the set of multi-dimensional features, an ethylene oligomerization catalyst structure as a most influential of the second iron ethylene oligomerization catalyst structures to the first K(C₁₂/C₁₀) values; and outputting the first

$$K ( \frac{{C}_{12}}{{C}_{10}} )$$

values and an indication that the ethylene oligomerization catalyst structure is the most influential of the second iron ethylene oligomerization catalyst structures to the first

$$K ( \frac{{C}_{12}}{{C}_{10}} )$$

values.

Statement 1.1 can include the method of statement 1, further comprising: inputting the set of multi-dimensional features to a set of machine learning models, wherein the set of models comprises the machine learning model configured to generate

$$K ( \frac{{C}_{12}}{{C}_{10}} )$$

values for the second iron ethylene oligomerization catalyst structures based on the multi-dimensional features; predicting, for each machine learning model in the set of machine learning models, respective

$$K ( \frac{{C}_{12}}{{C}_{10}} )$$

values; determining, for the respective

$$K ( \frac{{C}_{12}}{{C}_{10}} )$$

values, a respective root mean squared error (RMSE) over multiple iterations predicting the respective

$$K ( \frac{{C}_{12}}{{C}_{10}} )$$

values; and selecting the machine learning model from the set of machine learning models based on the machine learning model having the highest RMSE of the set of machine learning models.

Statement 1.2 can include the method for identifying catalyst structures comprising ligands for the iron-based oligomerization of ethylene of any of statements 1-1.1, wherein the set of multi-dimensional features comprises physical features which comprise catalyst loading, co-catalyst loading, co-catalyst type, ethylene pressure, reaction temperature, and time.

Statement 1.3 can include the method for identifying catalyst structures comprising ligands for the iron-based oligomerization of ethylene of any of statements 1-1.2, wherein the first iron ethylene oligomerization catalyst structures comprise an Fe metal center coordinated with N, O, S, and/or P ligands.

Statement 1.4 can include the method for identifying catalyst structures comprising ligands for the iron-based oligomerization of ethylene of any of statements 1-1.3, wherein the first iron ethylene oligomerization catalyst structures comprise an Fe metal center and pyridine-bisimine, α-diimine, phenanthroline, and iminopyridine ligands.

Statement 1.5 can include the method for identifying catalyst structures comprising ligands for the iron-based oligomerization of ethylene of any of statements 1-1.4, wherein the first iron ethylene oligomerization catalyst structures comprise the following structures:

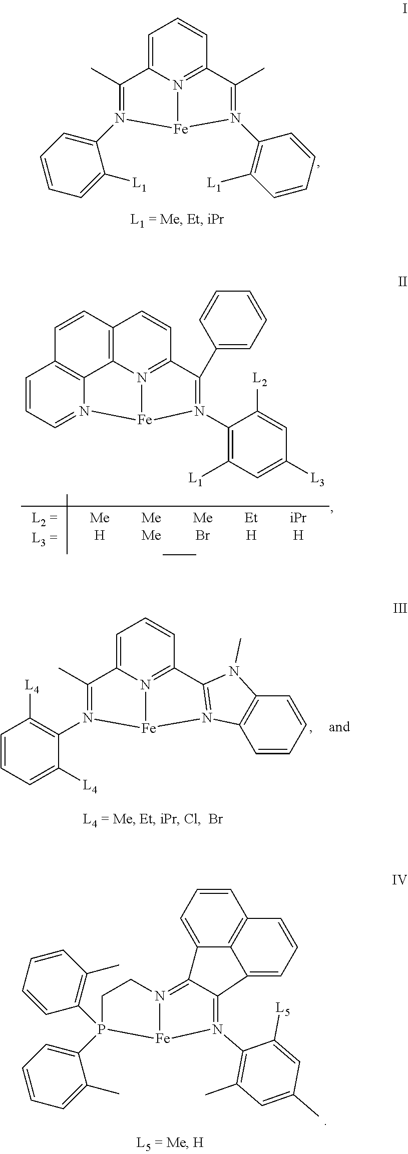

Statement 1.6 can include the method for identifying catalyst structures comprising ligands for the iron-based oligomerization of ethylene of statement 1.5, wherein the connective steric factors comprise the depth (depth_C₆) of C₆H₄(L₁) of structure I, C₆H₂(L₂)₂(L₃) of structure II, C₆H₃(L₄)₂ of structure III, and C₆H₂Me₂(L₅) of structure IV.

Statement 2 can include the method of any preceding statement, wherein the machine learning model is a random forest machine learning regressor model comprising decision trees, wherein each of the decision trees is trained on a subset of the second iron ethylene oligomerization catalyst structures.

Statement 2.1 can include the method of statement 2, wherein identifying the ethylene oligomerization catalyst structure as the most influential of the second iron ethylene oligomerization catalyst structures to the first

$$K ( \frac{{C}_{12}}{{C}_{10}} )$$

values is based on a proximity of theiron ethylene oligomerization catalyst structures to a subset of one or more second iron ethylene oligomerization catalyst structures selected from the second iron ethylene oligomerization catalyst structures.

Statement 3 can include the method of any preceding statement, wherein the set of multi-dimensional features are not based on information generated from quantum-chemical calculations.

Statement 3.1 can include the method of statement 3, wherein determining the set of multi-dimensional features comprises selecting the set of multi-dimensional features as a subset of multi-dimensional features based on their importance in predicting the

$$K ( \frac{{C}_{12}}{{C}_{10}} )$$

values.

Statement 3.2 can include the method of statement 3, wherein the set of multi-dimensional features comprises: an averaged molecular identifier on N atoms; a valence fifth order cluster Chi index; a subdivided surface area descriptor based on atomic logP and estimated accessible van der Waals surface area; a subdivided surface area descriptor based on atomic contribution to total polarizability of a ligand and estimated accessible van der Waals surface area; and a sum of E-state indices for C atoms in the ligand with one double bond and two single bonds.

Statement 4 can include a device for identifying catalyst structures comprising ligands for the iron-based oligomerization of ethylene, the device comprising memory coupled to at least one processor, the at least one processor configured to: determine a set of multi-dimensional features for first iron ethylene oligomerization catalyst structures, wherein the set of multi-dimensional features comprises physical features corresponding to reaction conditions, comprises molecular features, and comprises connective steric factors; input the set of multi-dimensional features to a machine learning model trained to predict

$$K ( \frac{{C}_{12}}{{C}_{10}} )$$

values measuring selectivity for propagation versus termination during oligomerization catalysis for second iron ethylene oligomerization catalyst structures based on the multi-dimensional features; predict, using the machine learning model and based on the set of multi-dimensional features, first

$$K ( \frac{{C}_{12}}{{C}_{10}} )$$

values; identify, using the machine learning model and based on the set of multi-dimensional features, an ethylene oligomerization catalyst structure as a most influential of the second iron ethylene oligomerization catalyst structures to the first

$$K ( \frac{{C}_{12}}{{C}_{10}} )$$

values; and output the first

$$K ( \frac{{C}_{12}}{{C}_{10}} )$$

values and an indication that the ethylene oligomerization catalyst structure is the most influential of the second iron ethylene oligomerization catalyst structures to the first

$$K ( \frac{{C}_{12}}{{C}_{10}} )$$

values.

Statement 4.1 can include the device of statement 4, wherein the at least one processor is further configured to: input the set of multi-dimensional features to a set of machine learning models, wherein the set of models comprises the machine learning model configured to generate

$$K ( \frac{{C}_{12}}{{C}_{10}} )$$

values for the second iron ethylene oligomerization catalyst structures based on the multi-dimensional features; predict, for each machine learning model in the set of machine learning models, respective

$$K ( \frac{{C}_{12}}{{C}_{10}} )$$

values; determine, for the respective

$$K ( \frac{{C}_{12}}{{C}_{10}} )$$

values, a respective root mean squared error (RMSE) over multiple iterations predicting the respective

$$K ( \frac{{C}_{12}}{{C}_{10}} )$$

values; and select the machine learning model from the set of machine learning models C₁₀ based on the machine learning model having the highest RMSE of the set of machine learning models.

Statement 4.2 can include the device for identifying catalyst structures comprising ligands for the iron-based oligomerization of ethylene of any of statements 4-4.1, wherein the set of multi-dimensional features comprises physical features which comprise catalyst loading, co-catalyst loading, co-catalyst type, ethylene pressure, reaction temperature, and time.

Statement 4.3 can include the device for identifying catalyst structures comprising ligands for the iron-based oligomerization of ethylene of any of statements 4-4.2, wherein the first iron ethylene oligomerization catalyst structures comprise an Fe metal center coordinated with N, O, S, and/or P ligands.

Statement 4.4 can include the device for identifying catalyst structures comprising ligands for the iron-based oligomerization of ethylene of any of statements 4-4.3, wherein the first iron ethylene oligomerization catalyst structures comprise an Fe metal center and pyridine-bisimin α-diimine, phenanthroline, and iminopyridine ligands.

Statement 4.5 can include the device for identifying catalyst structures comprising ligands for the iron-based oligomerization of ethylene of any of statements 4-4.4, wherein the first iron ethylene oligomerization catalyst structures comprise the following structures:

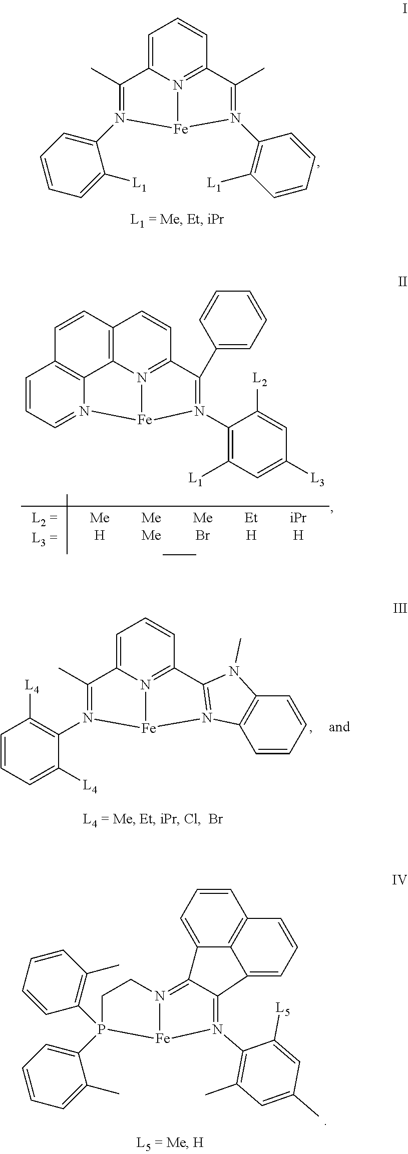

Statement 4.6 can include the device for identifying catalyst structures comprising ligands for the iron-based oligomerization of ethylene of statement 4.5, wherein the connective steric factors comprise the depth (depth_C₆) of C₆H₄(L₁) of structure I, C₆H₂(L₂)₂(L₃) of structure II, C₆H₃(L₄)₂ of structure III, and C₆H₂Me₂(L₅) of structure IV.

Statement 5 can include the device of any preceding statement, wherein the machine learning model is a random forest machine learning regressor model comprising decision trees, wherein each of the decision trees is trained on a subset of the second iron ethylene oligomerization catalyst structures.

Statement 5.1 can include the device of statement 5, wherein to identify the ethylene oligomerization catalyst structure as the most influential of the second iron ethylene oligomerization catalyst structures to the first

$$K ( \frac{{C}_{12}}{{C}_{10}} )$$

values is based on a proximity of the iron ethylene oligomerization catalyst structures to a subset of one or more second iron ethylene oligomerization catalyst structures selected from the second iron ethylene oligomerization catalyst structures.

Statement 6 can include the device of any preceding statement, wherein the set of multi-dimensional features are not based on information generated from quantum-chemical calculations.

Statement 6.1 can include the device of statement 6, wherein to determine the set of multi-dimensional features comprises selecting the set of multi-dimensional features as a subset of multi-dimensional features based on their importance in predicting the

$$K ( \frac{{C}_{12}}{{C}_{10}} )$$

values.

Statement 6.2 can include the device of statement 6, wherein the set of multi-dimensional features comprises: an averaged molecular identifier on N atoms; a valence fifth order cluster Chi index; a subdivided surface area descriptor based on atomic logP and estimated accessible van der Waals surface area; a subdivided surface area descriptor based on atomic contribution to total polarizability of a ligand and estimated accessible van der Waals surface area; a sum of E-state indices for C atoms in the ligand with one double bond and two single bonds; or any combination thereof.

Statement 7 can include a computer-readable medium storing instructions for identifying catalyst structures comprising ligands for the iron-based oligomerization of ethylene, that when executed by at least one processor causes the at least one processor to perform operations including: determining a set of multi-dimensional features for first iron ethylene oligomerization catalyst structures, wherein the set of multi-dimensional features comprises physical features corresponding to reaction conditions, comprises molecular features, and comprises connective steric factors; inputting the set of multi-dimensional features to a machine learning model trained to predict

$$K ( \frac{{C}_{12}}{{C}_{10}} )$$

values measuring selectivity for propagation versus termination during oligomerization catalysis for second iron ethylene oligomerization catalyst structures based on the multi-dimensional features; predicting, using the machine learning model and based on the set of multi-dimensional features, first

$$K ( \frac{{C}_{12}}{{C}_{10}} )$$

values; identifying, using the machine learning model and based on the set of multi-dimensional features, an ethylene oligomerization catalyst structure as a most influential of the second iron ethylene oligomerization catalyst structures to the first

$$K ( \frac{{C}_{12}}{{C}_{10}} )$$

values; and outputting the first

$$K ( \frac{{C}_{12}}{{C}_{10}} )$$

values and an indication that the ethylene oligomerization catalyst structure is the most influential of the second iron ethylene oligomerization catalyst structures to the first

$$K ( \frac{{C}_{12}}{{C}_{10}} )$$

values.

Statement 7.1 can include the computer-readable medium of statement 7, the instructions further comprising: inputting the set of multi-dimensional features to a set of machine learning models, wherein the set of models comprises the machine learning model configured to generate

$$K ( \frac{{C}_{12}}{{C}_{10}} )$$

values for the second iron ethylene oligomerization catalyst structures based on the multi-dimensional features; predicting, for each machine learning model in the set of machine learning models, respective

$$K ( \frac{{C}_{12}}{{C}_{10}} )$$

values; determining, for the respective

$$K ( \frac{{C}_{12}}{{C}_{10}} )$$

values, a respective root mean squared error (RMSE) over multiple iterations predicting the respective

$$K ( \frac{{C}_{12}}{{C}_{10}} )$$

values; and selecting the machine learning model from the set of machine learning models based on the machine learning model having the highest RMSE of the set of machine learning models.

Statement 7.2 can include the computer-readable medium for identifying catalyst structures comprising ligands for the iron-based oligomerization of ethylene of any of statements 7-7.1, wherein the set of multi-dimensional features comprises physical features which comprise catalyst loading, co-catalyst loading, co-catalyst type, ethylene pressure, reaction temperature, and time.

Statement 7.3 can include the computer-readable medium for identifying catalyst structures comprising ligands for the iron-based oligomerization of ethylene of any of statements 7-7.2, wherein the first iron ethylene oligomerization catalyst structures comprise an Fe metal center coordinated with N, O, S, and/or P ligands.

Statement 7.4 can include the computer-readable medium for identifying catalyst structures comprising ligands for the iron-based oligomerization of ethylene of any of statements 7-7.3, wherein the first iron ethylene oligomerization catalyst structures comprise an Fe metal center and pyridine-bisimine, α-diimine, phenanthroline, and iminopyridine ligands.

Statement 7.5 can include the computer-readable medium for identifying catalyst structures comprising ligands for the iron-based oligomerization of ethylene of any of statements 7-7.4, wherein the first iron ethylene oligomerization catalyst structures comprise the following structures:

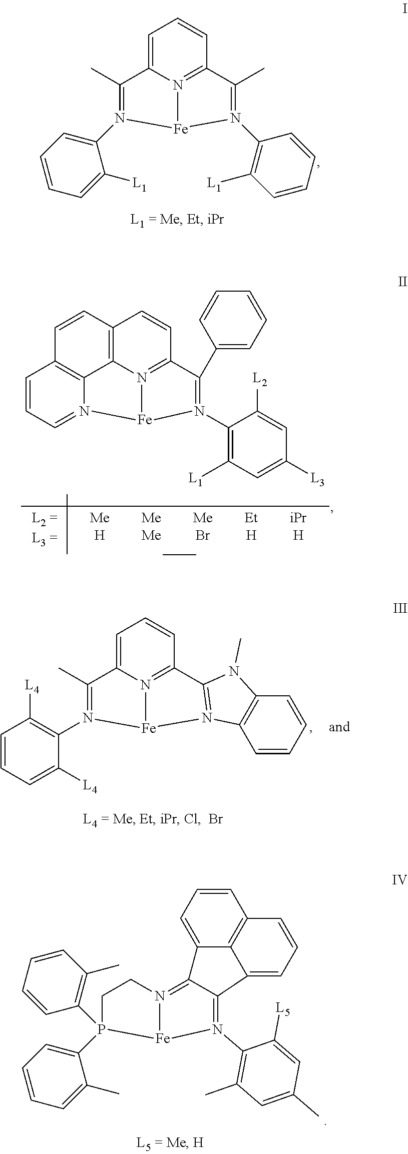

Statement 7.6 can include the computer-readable medium for identifying catalyst structures comprising ligands for the iron-based oligomerization of ethylene of statement 7.5, wherein the connective steric factors comprise the depth (depth_C₆) of C₆H₄(L₁) of structure I, C₆H₂(L₂)₂(L₃) of structure II, C₆H₃(L₄)₂ of structure III, and C₆H₂Me₂(L₅) of structure IV.

Statement 8 can include the computer-readable medium of any preceding statement, wherein the machine learning model is a random forest machine learning regressor model comprising decision trees, wherein each of the decision trees is trained on a subset of the second iron ethylene oligomerization catalyst structures.

Statement 8.1 can include the computer-readable medium of statement 8, wherein identifying the ethylene oligomerization catalyst structure as the most influential of the second iron ethylene oligomerization catalyst structures to the first

$$K ( \frac{{C}_{12}}{{C}_{10}} )$$

values is based on a proximity of a the iron ethylene oligomerization catalyst structures to a subset of one or more second iron ethylene oligomerization catalyst structures selected from the second iron ethylene oligomerization catalyst structures.

Statement 9 can include the computer-readable medium of any preceding statement, wherein the set of multi-dimensional features are not based on information generated from quantum-chemical calculations.

Statement 9.1 can include the computer-readable medium of statement 9, wherein determining the set of multi-dimensional features comprises selecting the set of multi-dimensional features as a subset of multi-dimensional features based on their importance in predicting the

$$K ( \frac{{C}_{12}}{{C}_{10}} )$$

values.

Statement 9.2 can include the computer-readable medium of statement 9, wherein the set of multi-dimensional features includes: an averaged molecular identifier on N atoms; a valence fifth order cluster Chi index; a subdivided surface area descriptor based on atomic logP and estimated accessible van der Waals surface area; a subdivided surface area descriptor based on atomic contribution to total polarizability of a ligand and estimated accessible van der Waals surface area; a sum of E-state indices for C atoms in the ligand with one double bond and two single bonds; or any combination thereof.

Statement 10. A method comprising: inputting a set of reaction conditions and a new iron ethylene oligomerization catalyst structure comprising a ligand to a random forest machine learning regressor model, wherein the random forest machine learning regressor model is trained on a data set comprising multi-dimensional features for tested iron ethylene oligomerization catalyst structures, wherein the multi-dimensional features comprise experimental K values, physical features, molecular features, and connective steric factors for each of the tested iron ethylene oligomerization catalyst structures; predicting, by the random forest machine learning regressor model, a predicted K value for the new iron ethylene oligomerization catalyst structure for the set of reaction conditions; and after predicting, experimentally determining an experimental K value for the new iron ethylene oligomerization catalyst structure under the set of reaction conditions.

Statement 11. The method of Statement 10, wherein the new iron ethylene oligomerization catalyst structure has at least one type of direct ligation to an Fe metal center in common with the tested iron ethylene oligomerization catalyst structures.

Statement 12. The method of Statement 10 or 11, wherein the physical features comprise catalyst loading, co-catalyst loading, co-catalyst type, ethylene pressure, reaction temperature, time, or a combination thereof.

Statement 13. The method of any one of Statements 10 to 12, wherein the molecular features comprise, for each of the tested iron ethylene oligomerization catalyst structures: an averaged molecular identifier on N atoms, a valence fifth order cluster Chi index, a subdivided surface area descriptor based on atomic logP and an estimated accessible van der Waals surface area, a subdivided surface area descriptor based on atomic contribution to total polarizability of a ligand and the estimated accessible van der Waals surface area, a sum of E-state indices for C atoms in the ligand with one double bond and two single bonds, or a combination thereof.

Statement 14. The method of any one of Statements 10 to 13, wherein the connective steric factors comprise, for each of the tested iron ethylene oligomerization catalyst structures: a size of a ligand arm branching from a main ligand core surrounding an Fe metal center of at least one of the tested iron ethylene oligomerization catalyst structures.

Statement 15. The method of any one of Statements 10 to 14, further comprising: determining a percentage difference between the experimental K value for the new iron ethylene oligomerization catalyst structure and the predicted K value for the new iron ethylene oligomerization catalyst structure.

Statement 16. The method of any one of Statements 10 to 15, wherein the experimental K value for the new iron ethylene oligomerization catalyst structure is within an 11% difference of the predicted K value for the new iron ethylene oligomerization catalyst structure.

Statement 17. The method of any one of Statements 10 to 16, further comprising: oligomerizing ethylene using the new iron ethylene oligomerization catalyst structure.

Statement 18A. The method of any one of Statements 10 to 17, wherein the tested iron ethylene oligomerization catalyst structures comprise an Fe metal center coordinated with a ligand selected from a N-containing ligand, an O-containing ligand, a S-containing ligand, a P-containing ligand, or a combination thereof.

Statement 18B. The method of any one of Statements 10 to 18A, wherein the new iron ethylene oligomerization catalyst structure comprises an Fe metal center coordinated with a ligand selected from a N-containing ligand, an O-containing ligand, a S-containing ligand, a P-containing ligand, or a combination thereof.

Statement 18C. The method of any one of Statements 10 to 18B, wherein the tested iron ethylene oligomerization catalyst structures comprise an Fe metal center coordinated with N, O, S, and/or P ligands.

Statement 18D. The method of any one of Statements 10 to 18C, wherein the new iron ethylene oligomerization catalyst structure comprises an Fe metal center coordinated with N, O, S, and/or P ligands.

Statement 18E. The method of any one of Statements 10 to 18D, wherein the tested iron ethylene oligomerization catalyst structures comprise an Fe metal center and pyridine-bisimine, a-diimine, phenanthroline, and iminopyridine ligands.

Statement 18F. The method of any one of Statements 10 to 18E, wherein the new iron ethylene oligomerization catalyst structure comprises an Fe metal center and pyridine-bisimine, α-diimine, phenanthroline, and iminopyridine ligands.

Statement 18G. The method of any one of Statements 10 to 18F, wherein the tested iron ethylene oligomerization catalyst structures comprise the following structures:

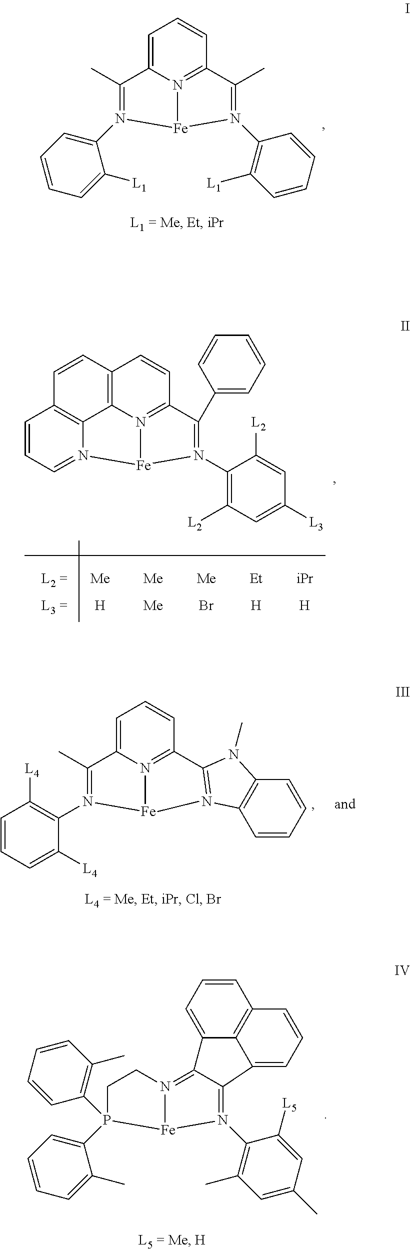

Statement 18H. The method of any one of Statements 10 to 18G, wherein the connective steric factors comprise the depth (depth_C₆) of C₆H₄(L₁) of structure I, C₆H₂(L₂)₂(L₃) of structure II, C₆H₃(L₄)₂ of structure III, and C₆H₂Me₂(L₅) of structure IV.

Statement 19. The method of any one of Statements 10 to 18H, wherein the ligand is a pyridine-bisimine ligand, an α-diimine ligand, a phenanthroline ligand, an iminopyridine ligand, or a combination thereof.

Statement 20. The method of any one of Statements 10 to 19, wherein the experimental K values for each of the tested iron ethylene oligomerization catalyst structures is an experimental

$$K ( \frac{{C}_{12}}{{C}_{10}} )$$

value or an experimental

$$K ( \frac{{C}_{14}}{{C}_{12}} )$$

value for each of the tested iron ethylene oligomerization catalyst structures.

Statement 21. The method of any one of Statements 10 to 20, wherein the predicted K value for the new iron ethylene oligomerization catalyst structure is a predicted

$$K ( \frac{{C}_{12}}{{C}_{10}} )$$

value or a predicted

$$K ( \frac{{C}_{14}}{{C}_{12}} )$$

value for the new iron ethylene oligomerization catalyst structure.

Statement 22. The method of any one of Statements 10 to 21, wherein the experimental K value for the new iron ethylene oligomerization catalyst structure is an experimental

$$K ( \frac{{C}_{12}}{{C}_{10}} )$$

value or an experimental

$$K ( \frac{{C}_{14}}{{C}_{12}} )$$

value for the new iron ethylene oligomerization catalyst structure.

Statement 23. The method of any one of Statements 10 to 22, wherein the predicted K value is predicted at a sub-kcal/mol accuracy.

Statement 24. The method of any one of Statements 10 to 23, wherein the multi-dimensional features are not based on information generated from quantum-chemical calculations.

Statement 25. A system comprising: a device comprising memory coupled to at least one processor, the memory having instructions that cause the at least one processor to: input a set of reaction conditions and a new iron ethylene oligomerization catalyst structure comprising a ligand to a random forest machine learning regressor model, wherein the random forest machine learning regressor model is trained on a data set comprising multi-dimensional features for tested iron ethylene oligomerization catalyst structures, wherein the multi-dimensional features comprise experimental K values, physical features, molecular features, and connective steric factors for each of the tested iron ethylene oligomerization catalyst structures; run the random forest machine learning regressor model to predict a predicted K value for the new iron ethylene oligomerization catalyst structure under the set of reaction conditions, wherein the predicted K value is obtained before an experimental K value is obtained for the new iron ethylene oligomerization catalyst structure.

Statement 26. The system of Statement 25, wherein:
the physical features comprise catalyst loading, co-catalyst loading, co-catalyst type, ethylene pressure, reaction temperature, time, or a combination thereof;
the molecular features comprise, for each of the tested iron ethylene oligomerization catalyst structures: an averaged molecular identifier on N atoms, a valence fifth order cluster Chi index, a subdivided surface area descriptor based on atomic logP and an estimated accessible van der Waals surface area, a subdivided surface area descriptor based on atomic contribution to total polarizability of a ligand and the estimated accessible van der Waals surface area, a sum of E-state indices for C atoms in the ligand with one double bond and two single bonds, or a combination thereof;
the connective steric factors comprise, for each of the tested iron ethylene oligomerization catalyst structures: a size of a ligand arm branching from a main ligand core surrounding an Fe metal center of at least one of the tested iron ethylene oligomerization catalyst structures; or
a combination of the above.

Statement 27A. The system of Statement 25 or 26, wherein: the predicted K value is predicted at a sub-kcal/mol accuracy.

Statement 27B. The system of any one of Statements 25 to 27A, wherein the multi-dimensional features are not based on information generated from quantum-chemical calculations.

Statement 28. The system of any one of Statements 25 to 27B, further comprising: an oligomerization reactor used to determine an experimental K value for the new iron ethylene oligomerization catalyst structure under the set of reaction conditions, after the precited K value is obtained.

Statement 29. The system of any one of Statements 25 to 28, wherein the instructions on the memory of the device cause the at least one processor to: determine a percentage difference between the experimental K value for the new iron ethylene oligomerization catalyst structure and the predicted K value for the new iron ethylene oligomerization catalyst structure.

Statement 30. A computer-readable medium storing instructions stored thereon, that when executed by at least one processor causes the at least one processor to perform operations including: input a set of reaction conditions and a new iron ethylene oligomerization catalyst structure comprising a ligand to a random forest machine learning regressor model, wherein the random forest machine learning regressor model is trained on a data set comprising multi-dimensional features for tested iron ethylene oligomerization catalyst structures, wherein the multi-dimensional features comprise experimental K values, physical features, molecular features, and connective steric factors for each of the tested iron ethylene oligomerization catalyst structures; run the random forest machine learning regressor model to predict a predicted K value for the new iron ethylene oligomerization catalyst structure under the set of reaction conditions, wherein the predicted K value is obtained before an experimental K value is obtained for the new iron ethylene oligomerization catalyst structure.

Embodiments of the present disclosure include various steps, which are described in this specification. The steps can be performed by hardware components or can be embodied in machine-executable instructions, which may be used to cause a general-purpose or special-purpose processor programmed with the instructions to perform the steps. Alternatively, the steps can be performed by a combination of hardware, software and/or firmware.

Various modifications and additions can be made to the exemplary embodiments discussed without departing from the scope of the present disclosure. For example, while the embodiments described above refer to particular features, the scope of this technology also includes embodiments having different combinations of features and embodiments that do not include all of the described features. Accordingly, the scope of the present disclosure is intended to embrace all such alternatives, modifications, and variations together with all equivalents thereof.

What is claimed:

1. A method comprising:
inputting a set of reaction conditions and a new iron ethylene oligomerization catalyst structure comprising a ligand to a random forest machine learning regressor model, wherein the random forest machine learning regressor model is trained on a data set comprising multi-dimensional features for tested iron ethylene oligomerization catalyst structures, wherein the multi-dimensional features comprise experimental K values, physical features, molecular features, and connective steric factors for each of the tested iron ethylene oligomerization catalyst structures;
predicting, by the random forest machine learning regressor model, a predicted K value for the new iron ethylene oligomerization catalyst structure for the set of reaction conditions; and
after predicting, experimentally determining an experimental K value for the new iron ethylene oligomerization catalyst structure under the set of reaction conditions.

2. The method of claim 1, wherein the new iron ethylene oligomerization catalyst structure has at least one type of direct ligation to an Fe metal center in common with the tested iron ethylene oligomerization catalyst structures.

3. The method of claim 1, wherein the physical features comprise catalyst loading, co-catalyst loading, co-catalyst type, ethylene pressure, reaction temperature, time, or a combination thereof.

4. The method of claim 1, wherein the molecular features comprise, for each of the tested iron ethylene oligomerization catalyst structures: an averaged molecular identifier on N atoms, a valence fifth order cluster Chi index, a subdivided surface area descriptor based on atomic logP and an estimated accessible van der Waals surface area, a subdivided surface area descriptor based on atomic contribution to total polarizability of a ligand and the estimated accessible van der Waals surface area, a sum of E-state indices for C atoms in the ligand with one double bond and two single bonds, or a combination thereof.

5. The method of claim 1, wherein the connective steric factors comprise, for each of the tested iron ethylene oligomerization catalyst structures: a size of a ligand arm branching from a main ligand core surrounding an Fe metal center of at least one of the tested iron ethylene oligomerization catalyst structures.

6. The method of claim 1, further comprising:
determining a percentage difference between the experimental K value for the new iron ethylene oligomerization catalyst structure and the predicted K value for the new iron ethylene oligomerization catalyst structure.

7. The method of claim 6, wherein the experimental K value for the new iron ethylene oligomerization catalyst structure is within an 11% difference of the predicted K value for the new iron ethylene oligomerization catalyst structure.

8. The method of claim 7, further comprising:
oligomerizing ethylene using the new iron ethylene oligomerization catalyst structure.

9. The method of claim 1, wherein the tested iron ethylene oligomerization catalyst structures comprise a Fe metal center coordinated with a ligand selected from a N-containing ligand, an O-containing ligand, a S-containing ligand, a P-containing ligand, or a combination thereof.

10. The method of claim 9, wherein the ligand is a pyridine-bisimine ligand, a α-diimine ligand, a phenanthroline ligand, a iminopyridine ligand, or a combination thereof.

11. The method of claim 1, wherein the experimental K values for each of the tested iron ethylene oligomerization catalyst structures is an experimental
$$K ( \frac{{C}_{12}}{{C}_{10}} )$$
value or an experimental
$$K ( \frac{{C}_{14}}{{C}_{12}} )$$
value for each of the tested iron ethylene oligomerization catalyst structures.

12. The method of claim 11, wherein the predicted K value for the new iron ethylene oligomerization catalyst structure is a predicted
$$K ( \frac{{C}_{12}}{{C}_{10}} )$$
value or a predicted
$$K ( \frac{{C}_{14}}{{C}_{12}} )$$
value for the new iron ethylene oligomerization catalyst structure.

13. The method of claim 12, wherein the experimental K value for the new iron ethylene oligomerization catalyst structure is an experimental
$$K ( \frac{{C}_{12}}{{C}_{10}} )$$
value or an experimental
$$K ( \frac{{C}_{14}}{{C}_{12}} )$$
value for the new iron ethylene oligomerization catalyst structure.

14. The method of claim 1, wherein the predicted K value is predicted at a sub-kcal/mol accuracy.

15. The method of claim 1, wherein the multi-dimensional features are not based on information generated from quantum-chemical calculations.

16. A system comprising:
a device comprising memory coupled to at least one processor, the memory having instructions that cause the at least one processor to:
input a set of reaction conditions and a new iron ethylene oligomerization catalyst structure comprising a ligand to a random forest machine learning regressor model, wherein the random forest machine learning regressor model is trained on a data set comprising multi-dimensional features for tested iron ethylene oligomerization catalyst structures, wherein the multi-dimensional features comprise experimental K values, physical features, molecular features, and connective steric factors for each of the tested iron ethylene oligomerization catalyst structures; and
run the random forest machine learning regressor model to predict a predicted K value for the new iron ethylene oligomerization catalyst structure under the set of reaction conditions, wherein the predicted K value is obtained before an experimental K value is obtained for the new iron ethylene oligomerization catalyst structure.

17. The system of claim 16, wherein:
the physical features comprise catalyst loading, co-catalyst loading, co-catalyst type, ethylene pressure, reaction temperature, time, or a combination thereof;
the molecular features comprise, for each of the tested iron ethylene oligomerization catalyst structures: an averaged molecular identifier on N atoms, a valence fifth order cluster Chi index, a subdivided surface area descriptor based on atomic logP and an estimated accessible van der Waals surface area, a subdivided surface area descriptor based on atomic contribution to total polarizability of a ligand and the estimated accessible van der Waals surface area, a sum of E-state indices for C atoms in the ligand with one double bond and two single bonds, or a combination thereof; and
the connective steric factors comprise, for each of the tested iron ethylene oligomerization catalyst structures: a size of a ligand arm branching from a main ligand core surrounding an Fe metal center of at least one of the tested iron ethylene oligomerization catalyst structures.

18. The system of claim 16, wherein:
the predicted K value is predicted at a sub-kcal/mol accuracy; or
the multi-dimensional features are not based on information generated from quantum-chemical calculations.

19. The system of claim 16, further comprising:
an oligomerization reactor used to determine an experimental K value for the new iron ethylene oligomerization catalyst structure under the set of reaction conditions, after the predicted K value is obtained.

20. The system of claim 19, wherein the instructions on the memory of the device cause the at least one processor to:
determine a percentage difference between the experimental K value for the new iron ethylene oligomerization catalyst structure and the predicted K value for the new iron ethylene oligomerization catalyst structure.
